SEQUENCE LISTING (80 sequences)

SEQ ID NO: 1 - DNA, 20 bp, Artificial Sequence
  Synthetic Polynucleotide
  misc_feature (1)..(3): Modified with a phosphorothioate bond
  misc_feature (14)..(20): Modified with a phosphorothioate bond
gggggacgatcgtcgggggg

SEQ ID NO: 2 - DNA, 24 bp, Artificial Sequence
  Synthetic Polynucleotide
  misc_feature (1)..(24): Modified with a phosphorothioate bond
tcgtcgttttgtcgttttgtcgtt

SEQ ID NO: 3 - DNA, 25 bp, Artificial Sequence
  Synthetic Polynucleotide
  misc_feature (1)..(25): Modified with a phosphorothioate bond
  misc_feature (25)..(25): Modified with /FluorT/
tcgtcgttttgtcgttttgtcgtrt

SEQ ID NO: 4 - DNA, 20 bp, Artificial Sequence
  Synthetic Polynucleotide
  misc_feature (1)..(20): Modified with a phosphorothioate bond
tccatgacgttcctgacgtt

SEQ ID NO: 5 - DNA, 22 bp, Artificial Sequence
  Synthetic Polynucleotide
  misc_feature (1)..(22): Modified with a phosphorothioate bond
tcgtcgttttcggcgcgcgccg

SEQ ID NO: 6 - DNA, 23 bp, Artificial Sequence
  Synthetic Polynucleotide
  misc_feature (1)..(23): Modified with a phosphorothioate bond
cgtcgttttgtcgttttgtcgtt

SEQ ID NO: 7 - DNA, 22 bp, Artificial Sequence
  Synthetic Polynucleotide
  misc_feature (1)..(22): Modified with a phosphorothioate bond
gtcgttttgtcgttttgtcgtt

SEQ ID NO: 8 - DNA, 20 bp, Artificial Sequence
  Synthetic Polynucleotide
  misc_feature (1)..(20): Modified with a phosphorothioate bond
cgttttgtcgttttgtcgtt

SEQ ID NO: 9 - DNA, 19 bp, Artificial Sequence
  Synthetic Polynucleotide
  misc_feature (1)..(19): Modified with a phosphorothioate bond
gttttgtcgttttgtcgtt

SEQ ID NO: 10 - DNA, 14 bp, Artificial Sequence
  Synthetic Polynucleotide
  misc_feature (1)..(14): Modified with a phosphorothioate bond
gtcgttttgtcgtt

SEQ ID NO: 11 - DNA, 13 bp, Artificial Sequence
  Synthetic Polynucleotide
  misc_feature (1)..(13): Modified with a phosphorothioate bond
tcgttttgtcgtt

SEQ ID NO: 12 - DNA, 12 bp, Artificial Sequence
  Synthetic Polynucleotide
  misc_feature (1)..(12): Modified with a phosphorothioate bond
cgttttgtcgtt

SEQ ID NO: 13 - DNA, 11 bp, Artificial Sequence
  Synthetic Polynucleotide
  misc_feature (1)..(11): Modified with a phosphorothioate bond
gttttgtcgtt

SEQ ID NO: 14 - DNA, 12 bp, Artificial Sequence
  Synthetic Polynucleotide
  misc_feature (1)..(12): Modified with a phosphorothioate bond
tcgtcgtttttt

SEQ ID NO: 15 - DNA, 12 bp, Artificial Sequence
  Synthetic Polynucleotide
  misc_feature (1)..(12): Modified with a phosphorothioate bond
tcgacgtttttt

SEQ ID NO: 16 - DNA, 14 bp, Artificial Sequence
  Synthetic Polynucleotide
  misc_feature (1)..(14): Modified with a phosphorothioate bond
tcgtttttttcgtt

SEQ ID NO: 17 - DNA, 14 bp, Artificial Sequence
  Synthetic Polynucleotide
  misc_feature (1)..(14): Modified with a phosphorothioate bond
tcgttttttttttt

SEQ ID NO: 18 - DNA, 14 bp, Artificial Sequence
  Synthetic Polynucleotide
  misc_feature (1)..(14): Modified with a phosphorothioate bond
ttttttcgtttttt

SEQ ID NO: 19 - DNA, 16 bp, Artificial Sequence
  Synthetic Polynucleotide
  misc_feature (1)..(16): Modified with a phosphorothioate bond
tcgtcgttcgtcgtta

SEQ ID NO: 20 - DNA, 21 bp, Artificial Sequence
  Synthetic Polynucleotide
  misc_feature (1)..(21): Modified with a phosphorothioate bond
tcgtcgttcgttcgttcgtta

SEQ ID NO: 21 - DNA, 24 bp, Artificial Sequence
  Synthetic Polynucleotide
  misc_feature (1)..(24): Modified with a phosphorothioate bond
tcgtcgtcgttcgtcgacgaacga

SEQ ID NO: 22 - DNA, 22 bp, Artificial Sequence
  Synthetic Polynucleotide
  misc_feature (1)..(22): Modified with a phosphorothioate bond
tcgtcgttttcgcggcgccgcg

SEQ ID NO: 23 - DNA, 23 bp, Artificial Sequence
  Synthetic Polynucleotide
  misc_feature (1)..(23): Modified with a phosphorothioate bond
tcgtcgtcgttcgaacgacgacg

SEQ ID NO: 24 - DNA, 29 bp, Artificial Sequence
  Synthetic Polynucleotide
  misc_feature (1)..(29): Modified with a phosphorothioate bond
tcgtcgtcgtcgcgttttcgcgacgacgt

SEQ ID NO: 25 - DNA, 28 bp, Artificial Sequence
  Synthetic Polynucleotide
  misc_feature (1)..(28): Modified with a phosphorothioate bond
tcgtcgtcgtcgcggaacgcgacgacgt

SEQ ID NO: 26 - DNA, 21 bp, Artificial Sequence
  Synthetic Polynucleotide
  misc_feature (1)..(21): Modified with a phosphorothioate bond
tcgttttgtcgttttgtcgtt

SEQ ID NO: 27 - DNA, 18 bp, Artificial Sequence
  Synthetic Polynucleotide
  misc_feature (1)..(18): Modified with a phosphorothioate bond
ttttgtcgttttgtcgtt

SEQ ID NO: 28 - DNA, 17 bp, Artificial Sequence
  Synthetic Polynucleotide
  misc_feature (1)..(17): Modified with a phosphorothioate bond
tttgtcgttttgtcgtt

SEQ ID NO: 29 - DNA, 16 bp, Artificial Sequence
  Synthetic Polynucleotide
  misc_feature (1)..(16): Modified with a phosphorothioate bond
ttgtcgttttgtcgtt

SEQ ID NO: 30 - DNA, 15 bp, Artificial Sequence
  Synthetic Polynucleotide
  misc_feature (1)..(15): Modified with a phosphorothioate bond
tgtcgttttgtcgtt

SEQ ID NO: 31 - DNA, 10 bp, Artificial Sequence
  Synthetic Polynucleotide
  misc_feature (1)..(10): Modified with a phosphorothioate bond
ttttgtcgtt

SEQ ID NO: 32 - DNA, 12 bp, Artificial Sequence
  Synthetic Polynucleotide
  misc_feature (1)..(12): Modified with a phosphorothioate bond
tgctgctttttt

SEQ ID NO: 33 - DNA, 12 bp, Artificial Sequence
  Synthetic Polynucleotide
  misc_feature (1)..(11): Modified with a phosphorothioate bond
tcgtcgtttttt

SEQ ID NO: 34 - DNA, 12 bp, Artificial Sequence
  Synthetic Polynucleotide
  misc_feature (1)..(8): Modified with a phosphorothioate bond
tcgtcgtttttt

SEQ ID NO: 35 - DNA, 12 bp, Artificial Sequence
  Synthetic Polynucleotide
  misc_feature (1)..(11): Modified with a phosphorothioate bond
tgctgctttttt

SEQ ID NO: 36 - DNA, 12 bp, Artificial Sequence
  Synthetic Polynucleotide
  misc_feature (1)..(8): Modified with a phosphorothioate bond
tgctgctttttt

SEQ ID NO: 37 - DNA, 11 bp, Artificial Sequence
  Synthetic Polynucleotide
  misc_feature (1)..(11): Modified with a phosphorothioate bond
tgctgcttttt

SEQ ID NO: 38 - DNA, 11 bp, Artificial Sequence
  Synthetic Polynucleotide
  misc_feature (1)..(11): Modified with a phosphorothioate bond
tcgtcgttttt

SEQ ID NO: 39 - DNA, 20 bp, Artificial Sequence
  Synthetic Polynucleotide
  misc_feature (1)..(3): Modified with a phosphorothioate bond
  misc_feature (14)..(20): Modified with a phosphorothioate bond
  misc_feature (20)..(20): Modified with /isp18//isp18//3CholTEG/
gggggacgatcgtcgggggg

SEQ ID NO: 40 - DNA, 24 bp, Artificial Sequence
  Synthetic Polynucleotide
  misc_feature (1)..(24): Modified with a phosphorothioate bond
  misc_feature (24)..(24): Modified with /iSp18//iSp18//3CholTEG/
tcgtcgttttgtcgttttgtcgtt

SEQ ID NO: 41 - DNA, 23 bp, Artificial Sequence
  Synthetic Polynucleotide
  misc_feature (1)..(23): Modified with a phosphorothioate bond
  misc_feature (23)..(23): Modified with /FluorT//iSp18//iSp18//3CholTEG/
tcgtcgttttgtcgttttgtcgt

SEQ ID NO: 42 - DNA, 20 bp, Artificial Sequence
  Synthetic Polynucleotide
  misc_feature (1)..(20): Modified with a phosphorothioate bond
  misc_feature (20)..(20): Modified with /isp18//isp18//3CholTEG/
tccatgacgttcctgacgtt

SEQ ID NO: 43 - DNA, 22 bp, Artificial Sequence
  Synthetic Polynucleotide
  misc_feature (1)..(22): Modified with a phosphorothioate bond
  misc_feature (22)..(22): Modified with /isp18//isp18//3CholTEG/
tcgtcgttttcggcgcgcgccg

SEQ ID NO: 44 - DNA, 23 bp, Artificial Sequence
  Synthetic Polynucleotide
  misc_feature (1)..(23): Modified with a phosphorothioate bond
  misc_feature (23)..(23): Modified with /isp18//isp18//3CholTEG/
cgtcgttttgtcgttttgtcgtt

SEQ ID NO: 45 - DNA, 22 bp, Artificial Sequence
  Synthetic Polynucleotide
  misc_feature (1)..(22): Modified with a phosphorothioate bond
  misc_feature (22)..(22): Modified with /isp18//isp18//3CholTEG/
gtcgttttgtcgttttgtcgtt

SEQ ID NO: 46 - DNA, 20 bp, Artificial Sequence
  Synthetic Polynucleotide
  misc_feature (1)..(20): Modified with a phosphorothioate bond
  misc_feature (20)..(20): Modified with /isp18//isp18//3CholTEG/
cgttttgtcgttttgtcgtt

SEQ ID NO: 47 - DNA, 19 bp, Artificial Sequence
  Synthetic Polynucleotide
  misc_feature (1)..(19): Modified with a phosphorothioate bond
  misc_feature (19)..(19): Modified with /isp18//isp18//3CholTEG/
gttttgtcgttttgtcgtt

SEQ ID NO: 48 - DNA, 14 bp, Artificial Sequence
  Synthetic Polynucleotide
  misc_feature (1)..(14): Modified with a phosphorothioate bond
  misc_feature (14)..(14): Modified with /isp18//isp18//3CholTEG/
gtcgttttgtcgtt

SEQ ID NO: 49 - DNA, 13 bp, Artificial Sequence
  Synthetic Polynucleotide
  misc_feature (1)..(13): Modified with a phosphorothioate bond
  misc_feature (13)..(13): Modified with /isp18//isp18//3CholTEG/
tcgttttgtcgtt

SEQ ID NO: 50 - DNA, 12 bp, Artificial Sequence
  Synthetic Polynucleotide
  misc_feature (1)..(12): Modified with a phosphorothioate bond
  misc_feature (12)..(12): Modified with /isp18//isp18//3CholTEG/
cgttttgtcgtt

SEQ ID NO: 51 - DNA, 11 bp, Artificial Sequence
  Synthetic Polynucleotide
  misc_feature (1)..(11): Modified with a phosphorothioate bond
  misc_feature (11)..(11): Modified with /isp18//isp18//3CholTEG/
gttttgtcgtt

SEQ ID NO: 52 - DNA, 12 bp, Artificial Sequence
  Synthetic Polynucleotide
  misc_feature (1)..(12): Modified with a phosphorothioate bond
  misc_feature (12)..(12): Modified with /isp18//isp18//3CholTEG/
tcgtcgtttttt

SEQ ID NO: 53 - DNA, 12 bp, Artificial Sequence
  Synthetic Polynucleotide
  misc_feature (1)..(12): Modified with a phosphorothioate bond
  misc_feature (12)..(12): Modified with /isp18//isp18//3CholTEG/
tcgacgtttttt

SEQ ID NO: 54 - DNA, 14 bp, Artificial Sequence
  Synthetic Polynucleotide
  misc_feature (1)..(14): Modified with a phosphorothioate bond
  misc_feature (14)..(14): Modified with /isp18//isp18//3CholTEG/
tcgtttttttcgtt

SEQ ID NO: 55 - DNA, 14 bp, Artificial Sequence
  Synthetic Polynucleotide
  misc_feature (1)..(14): Modified with a phosphorothioate bond
  misc_feature (14)..(14): Modified with /isp18//isp18//3CholTEG/
tcgttttttttttt

SEQ ID NO: 56 - DNA, 14 bp, Artificial Sequence
  Synthetic Polynucleotide
  misc_feature (1)..(14): Modified with a phosphorothioate bond
  misc_feature (14)..(14): Modified with /isp18//isp18//3CholTEG/
ttttttcgtttttt

SEQ ID NO: 57 - DNA, 16 bp, Artificial Sequence
  Synthetic Polynucleotide
  misc_feature (1)..(16): Modified with a phosphorothioate bond
  misc_feature (16)..(16): Modified with /isp18//isp18//3CholTEG/
tcgtcgttcgtcgtta

SEQ ID NO: 58 - DNA, 21 bp, Artificial Sequence
  Synthetic Polynucleotide
  misc_feature (1)..(21): Modified with a phosphorothioate bond
  misc_feature (21)..(21): Modified with /isp18//isp18//3CholTEG/
tcgtcgttcgttcgttcgtta

SEQ ID NO: 59 - DNA, 24 bp, Artificial Sequence
  Synthetic Polynucleotide
  misc_feature (1)..(24): Modified with a phosphorothioate bond
  misc_feature (24)..(24): Modified with /isp18//isp18//3CholTEG/
tcgtcgtcgttcgtcgacgaacga

SEQ ID NO: 60 - DNA, 22 bp, Artificial Sequence
  Synthetic Polynucleotide
  misc_feature (1)..(22): Modified with a phosphorothioate bond
  misc_feature (22)..(22): Modified with /isp18//isp18//3CholTEG/
tcgtcgttttcgcggcgccgcg

SEQ ID NO: 61 - DNA, 23 bp, Artificial Sequence
  Synthetic Polynucleotide
  misc_feature (1)..(23): Modified with a phosphorothioate bond
  misc_feature (23)..(23): Modified with /isp18//isp18//3CholTEG/
tcgtcgtcgttcgaacgacgacg

SEQ ID NO: 62 - DNA, 29 bp, Artificial Sequence
  Synthetic Polynucleotide
  misc_feature (1)..(29): Modified with a phosphorothioate bond
  misc_feature (29)..(29): Modified with /isp18//isp18//3CholTEG/
tcgtcgtcgtcgcgttttcgcgacgacgt

SEQ ID NO: 63 - DNA, 28 bp, Artificial Sequence
  Synthetic Polynucleotide
  misc_feature (1)..(28): Modified with a phosphorothioate bond
  misc_feature (28)..(28): Modified with /isp18//isp18//3CholTEG/
tcgtcgtcgtcgcggaacgcgacgacgt

SEQ ID NO: 64 - DNA, 21 bp, Artificial Sequence
  Synthetic Polynucleotide
  misc_feature (1)..(21): Modified with a phosphorothioate bond
  misc_feature (21)..(21): Modified with /isp18//isp18//3CholTEG/
tcgttttgtcgttttgtcgtt

SEQ ID NO: 65 - DNA, 18 bp, Artificial Sequence
  Synthetic Polynucleotide
  misc_feature (1)..(18): Modified with a phosphorothioate bond
  misc_feature (18)..(18): Modified with /isp18//isp18//3CholTEG/
ttttgtcgttttgtcgtt

SEQ ID NO: 66 - DNA, 17 bp, Artificial Sequence
  Synthetic Polynucleotide
  misc_feature (1)..(17): Modified with a phosphorothioate bond
  misc_feature (17)..(17): Modified with /isp18//isp18//3CholTEG/
tttgtcgttttgtcgtt

SEQ ID NO: 67 - DNA, 16 bp, Artificial Sequence
  Synthetic Polynucleotide
  misc_feature (1)..(16): Modified with a phosphorothioate bond
  misc_feature (16)..(16): Modified with /isp18//isp18//3CholTEG/
ttgtcgttttgtcgtt

SEQ ID NO: 68 - DNA, 15 bp, Artificial Sequence
  Synthetic Polynucleotide
  misc_feature (1)..(15): Modified with a phosphorothioate bond
  misc_feature (15)..(15): Modified with /isp18//isp18//3CholTEG/
tgtcgttttgtcgtt

SEQ ID NO: 69 - DNA, 10 bp, Artificial Sequence
  Synthetic Polynucleotide
  misc_feature (1)..(10): Modified with a phosphorothioate bond
  misc_feature (10)..(10): Modified with /isp18//isp18//3CholTEG/
ttttgtcgtt

SEQ ID NO: 70 - DNA, 12 bp, Artificial Sequence
  Synthetic Polynucleotide
  misc_feature (1)..(12): Modified with a phosphorothioate bond
  misc_feature (12)..(12): Modified with /isp18//isp18//3CholTEG/
tgctgctttttt

SEQ ID NO: 71 - DNA, 12 bp, Artificial Sequence
  Synthetic Polynucleotide
  misc_feature (1)..(11): Modified with a phosphorothioate bond
  misc_feature (12)..(12): Modified with /isp18//isp18//3CholTEG/
tcgtcgtttttt

SEQ ID NO: 72 - DNA, 12 bp, Artificial Sequence
  Synthetic Polynucleotide
  misc_feature (1)..(8): Modified with a phosphorothioate bond
  misc_feature (12)..(12): Modified with /isp18//isp18//3CholTEG/
tcgtcgtttttt

SEQ ID NO: 73 - DNA, 12 bp, Artificial Sequence
  Synthetic Polynucleotide
  misc_feature (1)..(11): Modified with a phosphorothioate bond
  misc_feature (12)..(12): Modified with /isp18//isp18//3CholTEG/
tgctgctttttt

SEQ ID NO: 74 - DNA, 12 bp, Artificial Sequence
  Synthetic Polynucleotide
  misc_feature (1)..(8): Modified with a phosphorothioate bond
  misc_feature (12)..(12): Modified with /isp18//isp18//3CholTEG/
tgctgctttttt

SEQ ID NO: 75 - DNA, 11 bp, Artificial Sequence
  Synthetic Polynucleotide
  misc_feature (1)..(11): Modified with a phosphorothioate bond
  misc_feature (11)..(11): Modified with /isp18//isp18//3CholTEG/
tgctgcttttt

SEQ ID NO: 76 - DNA, 11 bp, Artificial Sequence
  Synthetic Polynucleotide
  misc_feature (1)..(11): Modified with a phosphorothioate bond
  misc_feature (11)..(11): Modified with /isp18//isp18//3CholTEG/
tcgtcgttttt

SEQ ID NO: 77 - DNA, 34 bp, Artificial Sequence
  Synthetic Polynucleotide
  misc_feature (3)..(27): n is a, c, g, t, or absent
  misc_feature (29)..(29): n is a or g
  misc_feature (30)..(30): n is a, g, or t
  misc_feature (33)..(34): n is c or t
tcnnnnnnnnnnnnnnnnnnnnnnnnntnncgnn

SEQ ID NO: 78 - DNA, 56 bp, Artificial Sequence
  Synthetic Polynucleotide
  misc_feature (1)..(25): n is a, c, g, t, or absent
  misc_feature (26)..(27): n is a, c, g, or t
  misc_feature (30)..(31): n is a, c, g, or t
  misc_feature (32)..(56): n is a, c, g, t, or absent
nnnnnnnnnnnnnnnnnnnnnnnnnnncgnnnnnnnnnnnnnnnnnnnnnnnnnnn

SEQ ID NO: 79 - DNA, 12 bp, Artificial Sequence
  Synthetic Polynucleotide
cgacgttcgtcg

SEQ ID NO: 80 - DNA, 13 bp, Artificial Sequence
  Synthetic Polynucleotide
cggcgccgtgccg

(19) United States
(12) Patent Application Publication
(10) Pub. No.: US 2022/0064649 A1
(43) Pub. Date: Mar. 3, 2022

(54) SPHERICAL NUCLEIC ACID TLR9 AGONISTS

(71) Applicant: Exicure, Inc., Chicago, IL (US)

(72) Inventors: Bart Anderson, Chicago, IL (US); Subbarao Nallagatla, Chicago, IL (US); Richard Kang, Wilmette, IL (US); Ekambar Kandimalla, Hopkinton, MA (US)

(73) Assignee: Exicure, Inc., Chicago, IL (US)

(21) Appl. No.: 17/231,896

(22) Filed: Apr. 15, 2021

Related U.S. Application Data
(63) Continuation of application No. 16/099,404, filed on Nov. 6, 2018, now abandoned.
(60) Provisional application No. 62/333,074, filed on May 6, 2016.

(51) Int. Cl. C12N 15/117; A61K 9/127; A61K 39/395; C07K 16/28

(52) U.S. Cl. CPC C12N 15/117; A61K 9/127; A61K 39/3955; C12N 2320/32; C07K 16/2827; C12N 2310/17; C12N 2310/315; C07K 16/2818

(57) ABSTRACT

Aspects of the invention relate to compositions of spherical nucleic acids (SNAs) composed of a liposome or lipoplex complex having an oligonucleotide shell with CpG oligonucleotides positioned on the exterior of the liposome or lipoplex. The invention also relates to methods of treating subjects and of inducing cytokine expression in a subject using the compositions described herein.

### RELATED APPLICATIONS

This application claims the benefit under 35 U.S.C. § 119(e) of U.S. provisional application Ser. No. 62/333,074, filed May 6, 2016, the entire contents of which is incorporated by reference herein.

### BACKGROUND OF INVENTION

Toll-like receptor (TLR) 9 is an endosomal receptor that recognizes unmethylated CG motifs (CpG) in DNA, stimulating Th1-type immune responses. Previously identified synthetic oligonucleotide TLR9 agonists are linear or branched oligonucleotides. Linear oligonucleotide TLR9 agonists are divided into three major classes based on a combination of sequence properties and biological effects. A-class CpG oligonucleotides have a central self-complementary region with a phosphodiester (PO) backbone and 5′ and 3′ terminal repeats ≥3 G with phosphorothioate (PS) linkages, and stimulate high levels of IFNα production but low NF-κB activation. B-class CpG oligonucleotides have a PS backbone, minimal self-complementarity, and stimulate low levels of IFNα but high NF-κB activation. C-class CpG oligonucleotides have a PS backbone and high self-complementarity, and induce intermediate levels of IFNα and NF-κB. Linear oligonucleotide TLR9 agonists have a stringent requirement of sequence motif and length for TLR9 activation, typically requiring 24 nucleotides in length and for B and C class oligonucleotides a 5′ TCG for optimal activation of human TLR9.

### SUMMARY OF INVENTION

In some aspects, the invention is a spherical nucleic acid (SNA) which includes a liposome or lipoplex complex having an oligonucleotide shell comprised of B-class CpG oligonucleotides positioned on the exterior of the liposome or lipoplex, wherein the B-class CpG oligonucleotides are 4-16 nucleotides in length and/or do not have a 5′TCG motif.

In one embodiment, the CpG oligonucleotides are 8-14 nucleotides in length. In another embodiment, the CpG oligonucleotides do not have a 5′TCG motif.

In some embodiments, the CpG oligonucleotides are attached to the liposome or lipoplex through an anchor group. In one embodiment the anchor group is a lipid anchor group. In another embodiment, the anchor group is cholesterol. In another embodiment, the anchor group is tocopherol which may be alpha-tocopherol, beta-tocopherol, gamma-tocopherol or delta-tocopherol. In another embodiment, the anchor group may be chosen from sterol, palmitoyl, dipalmitoyl, stearyl, distearyl, C16 alkyl chain, bile acids, cholic acid, taurocholic acid, deoxycholate, oleyl litocholic acid, oleoyl cholenic acid, glycolipids, phospholipids, sphingolipids, isoprenoids, such as steroids, vitamins, such as vitamin E, saturated fatty acids, unsaturated fatty acids, fatty acid esters, such as triglycerides, pyrenes, porphyrines, Texaphyrine, adamantane, acridines, biotin, coumarin, fluorescein, rhodamine, Texas-Red, digoxygenin, dimethoxytrityl, t-butyldimethylsilyl, t-butyldiphenylsilyl, cyanine dyes (e.g. Cy3 or Cy5), Hoechst 33258 dye, psoralen, and ibuprofen or other lipophilic moieties.

In other embodiments, the oligonucleotides of the oligonucleotide shell are oriented radially outwards. In some embodiments, the oligonucleotide shell has a density of 5-1,000 oligonucleotides per SNA. In another embodiment, the oligonucleotide shell has a density of 100-1,000 oligonucleotides per SNA. In a further embodiment, the oligonucleotide shell has a density of 500-1,000 oligonucleotides per SNA.

In some embodiments, the oligonucleotides have at least one internucleoside phosphorothioate linkage. In other embodiments, the oligonucleotides do not have an internucleoside phosphorothioate linkage. In another embodiment, the oligonucleotides have all internucleoside phosphorothioate linkages. In another embodiment, the oligonucleotides have at least one internucleoside phosphorothioate linkage that is stereo-enriched. In another embodiment, the oligonucleotides have all the internucleoside phosphorothioate linkage that are stereo-enriched. The stereo-enriched phosphorothioate linkage may be Rp diastereomer, or Sp diastereomer.

In some embodiments, the oligonucleotides have a length of 10 to 12 nucleotides.

In another embodiment, at least 25 percent of the oligonucleotides have 5′-termini exposed to the outside surface of the SNA. In other embodiments, all of the oligonucleotides of the oligonucleotide shell have 5′ termini exposed to the outside surface of the SNA. In some embodiments, at least 25 percent of the oligonucleotides of the oligonucleotide shell have 3′-termini exposed to the outside surface of the SNA.

In other aspects, the invention is a composition including a spherical nucleic acid (SNA) comprised of a liposome or lipoplex complex having an oligonucleotide shell comprised of CpG oligonucleotides positioned on the exterior of the liposome or lipoplex, wherein the composition stimulates significantly more cytokine production than a molar equivalent of linear CpG oligonucleotides of the same sequence.

In other aspects, the invention is a composition including a spherical nucleic acid (SNA) comprised of a liposome or lipoplex complex having an oligonucleotide shell comprised of non-traditional CpG oligonucleotides positioned on the exterior of the liposome or lipoplex.

In some embodiments, the cytokine is IL 6. In another embodiment, the cytokine is IL-12. In another embodiment, the cytokine may be one or more of interferon alpha (IFN-α), interferon gamma (IFN-γ), interleukin 8 (IL 8), IL 18, tumor necrosis factor (TNF) and other cytokines known to be expressed as part of Th1-type or Th2-type immune response.

In one embodiment, the cytokine production is in vitro. In another embodiment, the cytokine production is in vivo.

In some embodiments, the CpG oligonucleotides are B-class CpG oligonucleotides. The CpG oligonucleotides, in another embodiment, are C-class CpG oligonucleotides. In other embodiments, the CpG oligonucleotides are A-class CpG oligonucleotides. In further embodiments, the CpG oligonucleotides are a mixture of A-class CpG oligonucleotides, B-class CpG oligonucleotides and C-class CpG oligonucleotides.

In one embodiment, the CpG oligonucleotides are 4-16 nucleotides in length. In some embodiments, the CpG oligonucleotides do not have a 5′TCG motif.

In another embodiment, the CpG oligonucleotides are oriented radially outwards. In some embodiments, the oligonucleotide shell has a density of 5-1,000 oligonucleotides per SNA. In other embodiments, the oligonucleotide shell has a density of 100-1,000 oligonucleotides per SNA. In another embodiment, the oligonucleotide shell has a density of 500-1,000 oligonucleotides per SNA.

In some embodiments, the CpG oligonucleotides have at least one internucleoside phosphorothioate linkage. In another embodiment, the CpG oligonucleotides do not have an internucleoside phosphorothioate linkage. In other embodiments, the CpG oligonucleotides have all internucleoside phosphorothioate linkages.

In another embodiment, the CpG oligonucleotides have a length of 10 to 16 nucleotides.

In further embodiments, at least 25 percent of the CpG oligonucleotides have 5′-termini exposed to the outside surface of the SNA. In some embodiments, at least 25 percent of the CpG oligonucleotides have 3′-termini exposed to the outside surface of the SNA.

Another aspect of the present disclosure includes a composition, comprising a spherical nucleic acid (SNA) comprised of a liposome or lipoplex complex having an oligonucleotide shell comprised of an A-class CpG oligonucleotides positioned on the exterior of the liposome or lipoplex.

In other aspects, the invention includes a method for inducing cytokine expression in a subject comprising: administering to a subject an effective amount for inducing IL-6 or IL-12 expression of a composition comprising a spherical nucleic acid (SNA) of a liposome or lipoplex complex having an oligonucleotide shell comprised of CpG oligonucleotides positioned on the exterior of the liposome or lipoplex. In some embodiments, the SNA is an SNA or a composition described above.

The invention, in another aspect, includes a method for treating a subject comprising: administering to a subject an effective amount for treating the subject of a composition comprising a spherical nucleic acid (SNA) of a liposome or lipoplex complex having an oligonucleotide shell comprised of CpG oligonucleotides positioned on the exterior of the liposome or lipoplex. In some embodiments, the SNA is an SNA or a composition described above. In one embodiment, the subject has cancer. In another embodiment, the subject has an infectious disease. In other embodiments, the subject has an allergic disorder or inflammatory disorder.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 includes two graphs depicting oligonucleotide uptake in human peripheral blood mononuclear cells (PMBCs). The top graph shows the percentage of hPBMCs taking up oligonucleotides and the bottom graph shows the oligonucleotide uptake per cell in hPBMCs.

FIG. 2 includes two graphs showing the cytokine response in vivo after mice were subcutaneously injected with CpG oligonucleotides. IL-12p70 levels are shown in the top graph and IL-6 levels are shown in the bottom graph.

### DETAILED DESCRIPTION

Although CpG oligonucleotides have been found to be immunostimulatory, the specific oligonucleotides which have therapeutic benefit in vivo are limited to a relatively narrow range of oligonucleotides having a preferred length and specific structure. CpG oligonucleotides having lower in vitro activity typically do not have enough activity to generate a therapeutically meaningful immune response in vivo. These sub-optimal CpG oligonucleotides are typically less than 24 nucleotides in length and do not include critical motifs such as a 5′TCG. It has been discovered according to the invention that sub-optimal CpG oligonucleotides when formulated as a Spherical nucleic acid (SNA) can produce a therapeutic immune response in vivo.

Spherical nucleic acids (SNAs) consist of densely packed, radially oriented nucleic acids. This architecture gives them unique properties, enabling cellular uptake of SNAs mediated via scavenger receptors. Cellular uptake of SNAs is fast and efficient and leads to endosomal accumulation. It has been discovered that CpG oligonucleotides formulated in SNAs have better cellular uptake with more SNA-oligonucleotides taken up into cells than oligonucleotide alone.

The SNAs of the invention include CpG oligonucleotides. In some embodiments those CpG oligonucleotides are sub-optimal or non-traditional CpG oligonucleotides. A “non-traditional” CpG oligonucleotide is an oligonucleotide containing an unmethylated CpG motif that while immunostimulatory in vitro does not produce a sufficient immune response in vivo to have a therapeutic benefit. In some embodiments a non-traditional CpG oligonucleotide has a length of less than 24 nucleotides. In other embodiments a non-traditional CpG oligonucleotide has one or more missing structural features from a traditional A-class, B-class, or C-class CpG oligonucleotide.

The CpG oligonucleotides in some embodiments are shorter than known therapeutic oligonucleotides of 24 nucleotides in length. The oligonucleotides are preferably in the range of 4 to 20 nucleotides in length. Preferably the oligonucleotides are in the range of between 6 and 16 and in some embodiments between 8 and 12, 8 and 10, 10 and 12, 6 and 12, 4 and 14, or 6 and 10 nucleotides in size.

CpG oligonucleotides include, for instance, A-class, B-class and C-class immunostimulatory CpG oligonucleotides. As used herein, the term “immunostimulatory CpG nucleic acids” or “immunostimulatory CpG oligonucleotides” refers to any CpG-containing oligonucleotide that is capable of activating an immune cell. At least the C of the CpG dinucleotide is typically unmethylated. Immunostimulatory CpG oligonucleotides are described in a number of issued patents and published patent applications, including U.S. Pat. Nos. 6,194,388; 6,207,646; 6,218,371; 6,239,116; 6,339,068; 6,406,705; and 6,429,199, which are incorporated by reference herein.

In some embodiments the immunostimulatory oligonucleotides have a modified backbone such as a phosphorothioate (PS) backbone. In other embodiments the immunostimulatory oligonucleotides have a phosphodiester (PO) backbone. In yet other embodiments immunostimulatory oligonucleotides have a mixed PO and PS backbone.

The CpG oligonucleotides may be A-class oligonucleotides, B-class oligonucleotides, or C-class oligonucleotides. “A-class” CpG immunostimulatory nucleic acids have been described in published PCT application WO 01/22990. These nucleic acids are characterized by the ability to induce high levels of interferon-alpha while having minimal effects on B cell activation. The A class CpG immunostimulatory nucleic acid may contain a hexamer palindrome GACGTC, AGCGCT, or AACGTT described by Yamamoto and colleagues. Yamamoto S et al. J Immunol 148:4072-6 (1992). Traditional A-class oligonucleotides have poly-G rich 5′ and 3′ ends and a palindromic center region. Typically the nucleotides at the 5′ and 3′ ends have stabilized internucleotide linkages and the center palindromic region has phosphodiester linkages (chimeric). A non-traditional A-class oligonucleotide may lack one or more of the poly G ends and the palindromic center. Alternatively the non-traditional A-class oligonucleotide may have all phosphorothioate or all phosphodiester internucleotide linkages.

B class CpG immunostimulatory nucleic acids strongly activate human B cells but have minimal effects inducing interferon-α without further modification. Traditionally, the B-class oligonucleotides include the sequence 5′ TCN₁TX₁X₂CGX₃X₄ 3′ (SEQ ID NO: 77), wherein X₁ is G or A; X₂ is T, G, or A; X₃ is T or C and X₄ is T or C; and N is any nucleotide, and N, and N₂ are nucleic acid sequences of about 0-25 N's each. B-class CpG oligonucleotides that are typically fully phosphorothiated and include an unmethylated CpG dinucleotide within certain preferred base contexts are potent at activating B cells and plasmacytoid dendritic cells (pDCs) but are relatively weak in inducing IFN-α and NK cell activation. See, e.g., U.S. Pat. Nos. 6,194,388; 6,207,646; 6,214,806; 6,218,371; 6,239,116; and 6,339,068.

In one embodiment a non-traditional B class CpG oligonucleotide is represented by at least the formula:

5′ X₁X₂CGX₃X₄ 3′

wherein X₁, X₂, X₃, and X₄ are nucleotides. In one embodiment X₂ is adenine, guanine, or thymine. In another embodiment X₃ is cytosine, adenine, or thymine. In some embodiments the non-traditional B class CpG oligonucleotide lacks a 5′TCG.

In another embodiment the invention provides an isolated B class CpG oligonucleotide represented by at least the formula:

(SEQ ID NO: 78)
5′ N₁X₁X₂CGX₃X₄N₂ 3′

wherein X₁, X₂, X₃, and X₄ are nucleotides and N is any nucleotide and N₁ and N₂ are nucleic acid sequences composed of from about 0-25 N's each. In one embodiment X₁X₂ is a dinucleotide selected from the group consisting of: GpT, GpG, GpA, ApA, ApT, ApG, CpT, CpA, CpG, TpA, TpT, and TpG; and X₃X₄ is a dinucleotide selected from the group consisting of: TpT, ApT, TpG, ApG, CpG, TpC, ApC, CpC, TpA, ApA, and CpA. Preferably X₁X₂ is GpA or GpT and X₃X₄ is TpT. In other embodiments X₁ or X₂ or both are purines and X₃ or X₄ or both are pyrimidines or X₁X₂ is GpA and X₃ or X₄ or both are pyrimidines. In another preferred embodiment X₁X₂ is a dinucleotide selected from the group consisting of: TpA, ApA, ApC, ApG, and GpG. In yet another embodiment X₃X₄ is a dinucleotide selected from the group consisting of: TpT, TpA, TpG, ApA, ApG, GpA, and CpA. X₁X₂ in another embodiment is a dinucleotide selected from the group consisting of: TpT, TpG. ApT, GpC, CpC, CpT, TpC, GpT and CpG; X₃ is a nucleotide selected from the group consisting of A and T and X₄ is a nucleotide, but wherein when X₁X₂ is TpC, GpT, or CpG, X₃X₄ is not TpC, ApT or ApC. In some embodiments the non-traditional B class CpG oligonucleotide lacks a 5′TCG.

In another preferred embodiment the CpG oligonucleotide has the sequence 5′ TCN₁TX₁X₂CGX₃X₄ 3′ (SEQ ID NO: 77) and a length of less than 24 nucleotides. The CpG oligonucleotides of the invention in some embodiments include X₁X₂ selected from the group consisting of GpT, GpG, GpA and ApA and X₃X₄ is selected from the group consisting of TpT, CpT and TpC.

In some embodiments, one or more of the B-Class CpG oligonucleotide is 2-20, 5-20, 10-20, 15-20, 2-20, 5-30, 10-30, 15-30, 20-30, 25-30, 2-40, 5-40, 10-40, 15-40, 20-40, 25-40, 30-40, 35-40, 2-50, 5-50, 10-50, 15-50, 20-50, 25-50, 30-50, 35-50, 2-100, 5-100, 10-100, 15-100, 20-100, 25-100, 30-100, 35-100 nucleotides in length. In some embodiments, the one or more of the B-Class CpG oligonucleotide is 4-16 nucleotides in length.

The C class immunostimulatory nucleic acids contain at least two distinct motifs and have unique and desirable stimulatory effects on cells of the immune system. Some of these ODN have both a traditional “stimulatory” CpG sequence and a “GC-rich” or “B-cell neutralizing” motif. These combination motif nucleic acids have immune stimulating effects that fall somewhere between those effects associated with traditional “class B” CpG ODN, which are strong inducers of B cell activation and dendritic cell (DC) activation, and those effects associated A-class CpG ODN which are strong inducers of IFN-α and natural killer (NK) cell activation but relatively poor inducers of B-cell and DC activation. See Krieg A M et al. (1995) Nature 374:546-9; Ballas Z K et al. (1996) J Immunol 157:1840-5; Yamamoto S et al. (1992) J Immunol 148:4072-6. While preferred class B CpG ODN often have phosphorothioate backbones and preferred class A CpG ODN have mixed or chimeric backbones, the C class of combination motif immune stimulatory nucleic acids may have either stabilized, e.g., phosphorothioate, chimeric, or phosphodiester backbones, and in some preferred embodiments, they have semi-soft backbones, e.g. a phosphodiester internucleotide linkage between the C and G nucleotides and other internucleotide linkages have a phosphorothioate linkage.

The stimulatory domain or motif is defined by a formula: 5′ X₁DCGHX₂ 3′. D is a nucleotide other than C. C is cytosine. G is guanine. H is a nucleotide other than G.

X₁ and X₂ are any nucleic acid sequence 0 to 10 nucleotides long. X₁ may include a CG, in which case there is preferably a T immediately preceding this CG. In some embodiments DCG is TCG. X₁ is preferably from 0 to 6 nucleotides in length. In some embodiments X₂ does not contain any poly G or poly A motifs. In other embodiments the immunostimulatory nucleic acid has a poly-T sequence at the 5′ end or at the 3′ end. As used herein, “poly-A” or “poly-T” shall refer to a stretch of three or more consecutive A's or T's respectively, e.g., 5′ AAAA 3′ or 5′ TTTT 3′.

As used herein, “poly-G end” shall refer to a stretch of three or more consecutive G's, e.g., 5′ GGG 3′, occurring at the 5′ end or the 3′ end of a nucleic acid. As used herein, “poly-G nucleic acid” shall refer to a nucleic acid having the formula 5′ X₁X₂GGGX₃X₄ 3′ wherein X₁, X₂, X₃, and X₄ are nucleotides and preferably at least one of X₃ and X₄ is a G.

Some preferred designs for the B cell stimulatory domain under this formula comprise TTTTTCG, TCG, TTCG, TTTCG, TTTTCG, TCGT, TTCGT, TTTCGT, TCGTCGT.

The second motif of the nucleic acid is referred to as either P or N and is positioned immediately 5′ to X₁ or immediately 3′ to X₂.

N is a B-cell neutralizing sequence that begins with a CGG trinucleotide and is at least 10 nucleotides long. A B-cell neutralizing motif includes at least one CpG sequence in which the CG is preceded by a C or followed by a G (Krieg A M et al. (1998) Proc Natl Acad Sci USA 95:12631-12636) or is a CG containing DNA sequence in which the C of the CG is methylated. As used herein, “CpG” shall refer to a 5′ cytosine (C) followed by a 3′ guanine (G) and linked by a phosphate bond. At least the C of the 5′ CG 3′ must be unmethylated. Neutralizing motifs are motifs which has some degree of immunostimulatory capability when present in an otherwise non-stimulatory motif, but, which when present in the context of other immunostimulatory motifs serve to reduce the immunostimulatory potential of the other motifs.

P is a GC-rich palindrome containing sequence at least 10 nucleotides long. As used herein, “palindrome” and, equivalently, “palindromic sequence” shall refer to an inverted repeat, i.e., a sequence such as ABCDEE′D′C′B′A′ in which A and A′, B and B′, etc., are bases capable of forming the usual Watson-Crick base pairs. P may also be an interrupted palindrome, i.e., a sequence such as ABCDENNNNE′D′C′B′A′ in which A and A′, B and B′, etc., are bases capable of forming the usual Watson-Crick base pairs and N is any base.

As used herein, “GC-rich palindrome” shall refer to a palindrome having a base composition of at least two-thirds G's and C's. In some embodiments the GC-rich domain is preferably 3′ to the “B cell stimulatory domain”. In the case of a 10-base long GC-rich palindrome, the palindrome thus contains at least 8 G's and C's. In the case of a 12-base long GC-rich palindrome, the palindrome also contains at least 8 G's and C's. In the case of a 14-mer GC-rich palindrome, at least ten bases of the palindrome are G's and C's. In some embodiments the GC-rich palindrome is made up exclusively of G's and C's.

In some embodiments the GC-rich palindrome has a base composition of at least 81% G's and C's. In the case of such a 10-base long GC-rich palindrome, the palindrome thus is made exclusively of G's and C's. In the case of such a 12-base long GC-rich palindrome, it is preferred that at least ten bases (83%) of the palindrome are G's and C's. In some preferred embodiments, a 12-base long GC-rich palindrome is made exclusively of G's and C's. In the case of a 14-mer GC-rich palindrome, at least twelve bases (86%) of the palindrome are G's and C's. In some preferred embodiments, a 14-base long GC-rich palindrome is made exclusively of G's and C's. The C's of a GC-rich palindrome can be unmethylated or they can be methylated.

In general this domain has at least 3 Cs and Gs, more preferably 4 of each, and most preferably 5 or more of each. The number of Cs and Gs in this domain need not be identical. It is preferred that the Cs and Gs are arranged so that they are able to form a self-complementary duplex, or palindrome, such as CCGCGCGG. This may be interrupted by As or Ts, but it is preferred that the self-complementarity is at least partially preserved as for example in the motifs CGACGTTCGTCG (SEQ ID NO: 79) or CGGCGCCGTGCCG (SEQ ID NO: 80). When complementarity is not preserved, it is preferred that the non-complementary base pairs be TG. In a preferred embodiment there are no more than 3 consecutive bases that are not part of the palindrome, preferably no more than 2, and most preferably only 1. In some embodiments the GC-rich palindrome includes at least one CGG trimer, at least one CCG trimer, or at least one CGCG tetramer.

In some embodiments the non-traditional C class CpG oligonucleotide lacks any one or more of the above features of a traditional C-class oligonucleotide, include nucleotide sequence, preferred length or backbone modification.

Spherical nucleic acids (SNAs) are a class of well-defined macromolecules, formed by organizing nucleic acids radially around a nanoparticle core, i.e., an inorganic metallic core (Mirkin C A, et. al. (1996) A DNA-based method for rationally assembling nanoparticles into macroscopic materials. Nature 382(6592):607-609.). These structures exhibit the ability to enter cells without the need for auxiliary delivery vehicles or transfection reagents by engaging class A scavenger receptors (SR-A) and lipid rafts (Rosi N L, et. al. (2006) Oligonucleotide-modified gold nanoparticles for intracellular gene regulation. Science 312: 1027-1031; Patel P C, et al. (2010) Scavenger receptors mediate cellular uptake of polyvalent oligonucleotide-functionalized gold nanoparticles. Bioconjugate chemistry 21(12):2250-2256.). Once inside the cell, the nucleic acid components of traditional SNAs resist nuclease degradation, leading to longer intracellular lifetimes. Moreover, SNAs, due to their multi-functional chemical structures, have the ability to bind their targets in a multivalent fashion (Choi C H, et. al. (2013) Mechanism for the endocytosis of spherical nucleic acid nanoparticle conjugates. Proceedings of the National Academy of Sciences of the United States of America 110(19):7625-7630; Wu X A, et. al. (2014) Intracellular fate of spherical nucleic acid nanoparticle conjugates. Journal of the American Chemical Society 136(21):7726-7733).

It has been discovered herein that CpG oligonucleotides formulated as SNA lipid based delivery systems have enhanced therapeutic properties. CpG oligonucleotide SNAs have been developed according to the invention which incorporate lipid nanoparticles in a densely packed CpG oligonucleotide shell. These unique molecules can be used to efficiently deliver any type of therapeutic or diagnostic reagent to a cell, and in particular to cells in an efficient manner, resulting in enhanced therapeutic responses. The liposome or lipoplex with optional therapeutic agents incorporated therein can be functionalized into an SNA by inserting lipid-conjugated nucleic acids into its external surface. The resulting SNAs will contain lipids and the outer radially oriented nucleic acids. Molecules packaged in the aforementioned SNAs will be taken up into cells via scavenger receptor-mediated endocytosis, resulting in efficient and fast endosomal accumulation characteristic of other SNAs. Functionalizing the liposome/lipoplex as an SNA changes the route of uptake. Functionalizing the liposome/lipoplex as an SNA increases the efficiency, kinetics, or endosomal accumulation of the liposome/lipoplex. By functionalizing the surface of a liposome/lipoplex into a SNA, the route of cellular delivery is directed through scavenger receptors, enhancing endosomal uptake in vitro and in vivo.

The nanostructures of the invention are typically composed of an interchangeable nanoparticle core having a shell of oligonucleotides, which is formed by arranging CpG oligonucleotides such that they point radially outwards from the core. In some aspects, the nanoparticle core may be lipid nanoparticles. Alternatively, the nanoparticle core may be composed of niosomes. A hydrophobic (e.g. lipid) anchor group attached to either the 5′- or 3′-end of the oligonucleotide, depending on whether the oligonucleotides are arranged with the 5′- or 3′-end facing outward from the core preferably is used to embed the oligonucleotides in the lipid nanoparticle. The anchor acts to drive insertion into the lipid nanoparticle and to attach the oligonucleotides to the lipids.

Niosomes are vesicles formed from non-ionic surfactant oriented in a bilayer. Niosomes commonly have cholesterol added as an excipient, but other lipid-based and non-lipid-based constituents can also be included. Methods for preparation of niosomes are known in the art. In some embodiments polyethylene glycol (PEG) is included during or following niosome preparation. Niosome vesicles are structurally and functionally analogous to liposomes, but are based on non-ionic surfactant rather than lipid as the primary constituent. Common non-ionic surfactants used include sorbitans (spans) or polysorbates (tween); however, a wide variety of non-ionic surfactants can be used to prepare niosomes.

The lipid nanoparticle can be constructed from a wide variety of lipids known to those in the art to produce a liposome or lipoplex. A liposome is a structure composed of at least one lipid bilayer membrane that encloses an internal compartment. Liposomes may be characterized according to the membrane type and size. Small unilamellar vesicles (SUVs) have a single membrane and typically range between 0.02 and 0.05 pm in diameter; large unilamellar vesicles (LUVS) are typically larger than 0.05 pm. Oligolamellar large vesicles and multilamellar vesicles have multiple, usually concentric, membrane layers and are typically larger than 0.1 pm. Liposomes with several nonconcentric membranes, i.e., several smaller vesicles contained within a larger vesicle, are termed multivesicular vesicles.

The nanostructure of the invention may include a core. The core may be a hollow core, which has at least some space in the center region of a shell material. Hollow cores include liposomal cores.

A liposomal core as used herein refers to a centrally located core compartment formed by a component of the lipids or phospholipids that form a lipid bilayer. The lipid bilayer is composed of two layers of lipid molecules. Each lipid molecule in a layer is oriented substantially parallel to adjacent lipid bilayers, and two layers that form a bilayer have the polar ends of their molecules exposed to the aqueous phase and the non-polar ends adjacent to each other. The central aqueous region of the liposomal core may be empty or filled fully or partially with water, an aqueous emulsion, oligonucleotides, or other therapeutic or diagnostic agent.

A lipoplex, is a type of lipid nanoparticle which specifically incorporates nucleic acids in a lipid-nucleic acid complex. Lipoplexes, also referred to as nucleic acid lipid particles typically contain a cationic lipid or a non-cationic lipid and optionally a sterol and/or a lipid that prevents aggregation of the particle (e.g., a PEG-lipid conjugate). In some instances the lipoplex contains a cationic lipid, a non-cationic lipid, a sterol and a lipid.

Other lipids may be included in the lipid nanoparticle for a variety of purposes, such as to prevent lipid oxidation or to attach ligands onto lipid nanoparticle surface. Any of a number of lipids may be present, including amphipathic, neutral, cationic, and anionic lipids. Such lipids can be used alone or in combination. Additional components that may be present in a lipid nanoparticle include bilayer stabilizing components such as polyamide oligomers, peptides, proteins, detergents, lipid-derivatives, such as PEG coupled to phosphatidylethanolamine and PEG conjugated to ceramides. The lipid nanoparticles may also include one or more of a second amino lipid or cationic lipid, a neutral lipid, a sterol, and a lipid selected to reduce aggregation of lipid particles during formation, which may result from steric stabilization of particles which prevents charge-induced aggregation during formation. As used herein, the term “amino lipid” is meant to include those lipids having one or two fatty acid or fatty alkyl chains and an amino head group (including an alkylamino or dialkylamino group) that may be protonated to form a cationic lipid at physiological pH.

The lipid nanoparticle or niosome may have a mean diameter of about 10 nm to about 150 nm, more typically about 60 nm to about 130 nm, more typically about 70 nm to about 110 nm, most typically about 70 nm to about 90 nm. In certain embodiments, the diameter of the lipid nanoparticle or niosome is from 1 nm to about 250 nm in mean diameter, about 1 nm to about 240 nm in mean diameter, about 1 nm to about 230 nm in mean diameter, about 1 nm to about 220 nm in mean diameter, about 1 nm to about 210 nm in mean diameter, about 1 nm to about 200 nm in mean diameter, about 1 nm to about 190 nm in mean diameter, about 1 nm to about 180 nm in mean diameter, about 1 nm to about 170 in mean diameter, about 1 nm to about 160 nm in mean diameter, about 1 nm to about 150 nm in mean diameter, about 1 nm to about 140 nm in mean diameter, about 1 nm to about 130 nm in mean diameter, about 1 nm to about 120 nm in mean diameter, about 1 nm to about 110 nm in mean diameter, about 1 nm to about 100 nm in mean diameter, about 1 nm to about 90 nm in mean diameter, about 1 nm to about 80 nm in mean diameter, about 1 nm to about 70 nm in mean diameter, about 1 nm to about 60 nm in mean diameter, about 1 nm to about 50 nm in mean diameter, about 1 nm to about 40 nm in mean diameter, about 1 nm to about 30 nm in mean diameter, or about 1 nm to about 20 nm in mean diameter, or about 1 nm to about 10 nm in mean diameter.

The oligonucleotides may be positioned on the exterior of the lipid nanoparticle or niosome, within the walls of the core and/or in the center of the core. An oligonucleotide that is positioned on the core is typically referred to as coupled to the core. Coupled may be direct or indirect. In some embodiments at least 5, 10, 15, 25, 50, 75, 100, 200, 300, 400, 500, 600, 700, 800, 900, 1000, 5000 or 10,000 oligonucleotides or any range combination thereof are on the exterior of the core. In some embodiments, 1-1000, 10-500, 50-250, or 50-300 oligonucleotides are present on the surface.

The lipid nanoparticle may include a neutral lipid. The neutral lipid may be, for example, 1,2-dioleoyl-sn-glycero-3-phosphocholine (DOPC), 1,2-dimyristoyl-sn-phosphatidylcholine (DMPC), 1-palmitoyl-2-oleoyl-sn-phosphatidylcholine (POPC), 1,2-distearoyl-sn-glycero-3-phospho-(1′-rac-glycerol) (DSPG), 1,2-dioleoyl-sn-glycero-3-phospho-(1′-rac-glycerol) (DOPG), 1,2-distearoyl-sn-glycero-3-phosphocholine (DSPC), 1,2-dipalmitoyl-sn-glycero-3-phosphocholine (DPPC), 1,2-di-(9Z-octadecenoyl)-sn-glycero-3-phosphoethanolamine (DOPE), and 1,2-dihexadecanoyl-sn-glycero-3-phosphoethanolamine (DPPE), any related phosphatidylcholine or neutral lipids available from commercial vendors.

The lipid nanoparticle may include a cationic lipid. The cationic lipid may be, for example, N,N-dioleyl-N,N-dimethylammonium chloride (DODAC), N,N-distearyl-N,N-dimethylammonium bromide (DDAB), N-(1-(2,3-dioleoyloxy)propyl)-N,N,N-trimethylammonium chloride (DOTAP), N-(1-(2,3-dioleyloxy)propyl)-N,N,N-trimethylammonium chloride (DOTMA), N,N-dimethyl-2,3-dioleyloxy)propylamine (DODMA), 1,2-DiLinoleyloxy-N,N-dimethylaminopropane (DLinDMA), 1,2-Dilinolenyloxy-N,N-dimethylaminopropane (DLenDMA), 1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.C1), 1,2-Dilinoleoyl-3-trimethylaminopropane chloride salt (DLin-TAP.C1), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanedio (DOAP), 1,2-Dilinoleyloxo-3-(2-N,N-dimethylamino)ethoxypropane (DLin-EG-DMA), 1,2-Dilinolenyloxy-N,N-dimethylaminopropanc (DLinDMA), 2,2-Dilinoleyl-4-dimethylaminomethyl-[1,3]-dioxolane (DLin-K-DMA) or analogs thereof, (3aR,5s,6aS)—N,N-dimethyl-2,2-di((9Z,12Z)-octadeca-9,12-dienyl)tetrahydro-3aH-cyclopenta[d][1,3]dioxol-5-amine, (6Z,9Z,28Z,31Z)-heptatriaconta-6,9,28,31-tetraen-19-yl-4-(dimethylamino)butanoate, or a mixture thereof.

Other cationic lipids, which carry a net positive charge at about physiological pH, in addition to those specifically described above, may also be included in the lipid nanoparticle. Such cationic lipids include, but are not limited to, N,N-dioleyl-N,N-dimethylammonium chloride (“DODAC”); N-(2,3-dioleyloxy)propyl-N,N—N-triethylammonium chloride (“DOTMA”); N,N-distearyl-N,N-dimethylammonium bromide (“DDAB”); N-(2,3-dioleoyloxy)propyl)-N,N,N-trimethylammonium chloride (“DOTAP”); 1,2-Dioleyloxy-3-trimethylaminopropane chloride salt (“DOTAP.C1”); 3.beta.-(N—(N′,N′-dimethylaminoethane)-carbamoyl)cholesterol (“DC-Chol”), N-(1-(2,3-dioleyloxy)propyl)-N-2-(sperminecarboxamido)ethyl)-N,N-dimethyl-ammonium trifluoracetate (“DOSPA”), dioctadecylamidoglycyl carboxyspermine (“DOGS”), 1,2-dileoyl-sn-3-phosphoethanolamine (“DOPE”), 1,2-dioleoyl-3-dimethylammonium propane (“DODAP”), N,N-dimethyl-2,3-dioleyloxy)propylamine (“DODMA”), and N-(1,2-dimyristyloxyprop-3-yl)-N,N-dimethyl-N-hydroxyethyl ammonium bromide (“DMRIE”).

“Amphipathic lipids” refer to any suitable material, wherein the hydrophobic portion of the lipid material orients into a hydrophobic phase, while the hydrophilic portion orients toward the aqueous phase. Such compounds include, but are not limited to, phospholipids, aminolipids, and sphingolipids. Representative phospholipids include sphingomyelin, phosphatidylcholine, phosphatidylethanolamine, phosphatidylserine, phosphatidylinositol, phosphatidic acid, palmitoyloleoyl phosphatdylcholine, lysophosphatidylcholine, lysophosphatidylethanolamine, dipalmitoylphosphatidylcholine, dioleoylphosphatidylcholine, distearoylphosphatidylcholine, or dilinoleylphosphatidylcholine.

The lipid nanoparticles described in the invention may include varying ratios of cationic lipids, neutral lipids, sterols and PEG-modified lipids.

Therapeutic agents may be incorporated into the SNA. These include but are not limited to small molecules, proteins, nucleic acids, gases (e.g. NO), dyes, vitamins, nutrients, antibiotics, antifungals, and antivirals, chemotherapeutic agents, steroids, hormones, magnetic or paramagnetic particles, encapsulated therapeutic drugs, prodrugs or molecules, water soluble or water insoluble molecules, and proteins, including those that function in the endosome, especially the ones associated with endosomal storage diseases.

The liposome/lipoplex may also be constructed to contain other surface elements including but not limited to: aptamers, antibodies, proteins, peptides, lipid derivatives; small molecules, and magnetic/paramagnetic particles. These may be used for various purposes.

The oligonucleotide shell can be constructed from a wide variety of CpG oligonucleotides as described herein. Preferably CpG oligonucleotides are single-stranded deoxyribonucleotides. However, the oligonucleotides may also be ribonucleotides, and other single-stranded oligonucleotides incorporating one or a multiplicity of modifications known to those in the art, double-stranded deoxyribonucleotides, ribonucleotides, and other double-stranded oligonucleotides incorporating one or a multiplicity of modifications known to those in the art, oligonucleotide triplexes incorporating deoxyribonucleotides, ribonucleotides, or oligonucleotides that incorporate one or a multiplicity of modifications known to those in the art. These oligonucleotides may or may not be pre-complexed with proteins, polymers, or carriers including protamine.

In some embodiments, the oligonucleotide shell has a density of 25, 50, 75, 100, 200, 300, 400, 500, 600, 700, 800, 900, 1,000, 2,000, 3,000, 4,000, 5,000, 6,000, 7,000, 8,000, 9,000 or 10,000 oligonucleotides or any range combination thereof. In other embodiments, the oligonucleotide shell has a density of 1-10,000, 1-9,000, 1-8,000, 1-7,000, 1-6,000, 1-5,000, 1-4,000, 1-3,000, 1-2,000, 1-1,000, 5-10,000, 5-9,000, 5-8,000, 5-7,000, 5-6,000, 5-5,000, 5-4,000, 5-3,000, 5-2,000, 5-1,000, 100-10,000, 100-9,000, 100-8,000, 100-7,000, 100-6,000, 100-5,000, 100-4,000, 100-3,000, 100-2,000, 100-1,000, 500-10,000, 500-9,000, 500-8,000, 500-7,000, 500-6,000, 500-5,000, 500-4,000, 500-3,000, 500-2,000, 500-1,000, 10-10,000, 10-500, 50-10,000, 50-300, or 50-250.

In some embodiments, the oligonucleotides of the oligonucleotide shell are structurally identical oligonucleotides. In other embodiments, the oligonucleotides of the oligonucleotide shell have at least two structurally different oligonucleotides. In certain embodiments, the oligonucleotides of the oligonucleotide shell have 2-50, 2-40, 2-30, 2-20 or 2-10 different nucleotide sequences.

In some embodiments, at least 60%, 70%, 80%, 90%, 95%, 96%, 97% 98% or 99% of the oligonucleotides are positioned on the surface of the nanostructure.

In some embodiments, the oligonucleotides form an oligonucleotide shell. An oligonucleotide shell is formed when at least 10% of the available surface area of the exterior surface of a liposomal core includes an oligonucleotide. In some embodiments at least 20%, 30%, 40%, 50%, 60%, 70%, 80%, 90%, 95%, 96%, 97%, 98% or 99% of the available surface area of the exterior surface of the liposomal includes an oligonucleotide. The oligonucleotides of the oligonucleotide shell may be oriented in a variety of directions. In some embodiments the oligonucleotides are oriented radially outwards.

In some embodiments, at least 10% of the oligonucleotides in the oligonucleotide shell are attached to the nanoparticle through a lipid anchor group. The lipid anchor consists of a hydrophobic group that enables insertion and anchoring of the oligonucleotides or nucleic acids to the lipid membrane. In some embodiments, at least 20%, at least 30%, at least 40%, at least 50%, at least 60%, at least 70%, at least 80%, at least 90%, at least 95%, at least 99%, or 100% of the oligonucleotides in the oligonucleotide shell are attached to the lipid nanoparticle through a lipid anchor group. In some embodiments, the lipid anchor group is cholesterol. In other embodiments, the lipid anchor group is sterol, palmitoyl, dipalmitoyl, stearyl, distearyl, C16 alkyl chain, bile acids, cholic acid, taurocholic acid, deoxycholate, oleyl litocholic acid, oleoyl cholenic acid, glycolipids, phospholipids, sphingolipids, isoprenoids, such as steroids, vitamins, such as vitamin E, saturated fatty acids, unsaturated fatty acids, fatty acid esters or other lipids known in the art.

The terms “oligonucleotide” and “nucleic acid” are used interchangeably to mean multiple nucleotides (i.e., molecules comprising a sugar (e.g., ribose or deoxyribose) linked to a phosphate group and to an exchangeable organic base, which is either a substituted pyrimidine (e.g., cytosine (C), thymidine (T) or uracil (U)) or a substituted purine (e.g., adenine (A) or guanine (G)). Thus, the term embraces both DNA and RNA oligonucleotides. The terms shall also include polynucleosides (i.e., a polynucleotide minus the phosphate) and any other organic base containing polymer. Oligonucleotides can be obtained from existing nucleic acid sources (e.g., genomic or cDNA), but are preferably synthetic (e.g., produced by nucleic acid synthesis). The term “nucleoside” includes bases which are covalently attached to a sugar moiety, preferably ribose or deoxyribose. The term “nucleotide” includes nucleosides which further comprise a phosphate group or a phosphate analog.

Oligonucleotides associated with the invention can be modified such as at the sugar moiety, the phosphodiester linkage, and/or the base. As used herein, “sugar moieties” includes natural, unmodified sugars, including pentose, ribose and deoxyribose, modified sugars and sugar analogs. Modifications of sugar moieties can include replacement of a hydroxyl group with a halogen, a heteroatom, or an aliphatic group, and can include functionalization of the hydroxyl group as, for example, an ether, amine or thiol.

Modification of sugar moieties can include 2′-O-methyl nucleotides, which are referred to as “methylated.” In some instances, oligonucleotides associated with the invention may only contain modified or unmodified sugar moieties, while in other instances, oligonucleotides contain some sugar moieties that are modified and some that are not.

In some instances, modified nucleotides include sugar- or backbone-modified ribonucleotides or deoxyribonucleotides. Modified ribo or deoxyribonucleotides can contain a non-naturally occurring base such as uridines or cytidines modified at the 5′-position, e.g., 5′-(2-amino)propyl uridine and 5′-bromo uridine; adenosines and guanosines modified at the 8-position, e.g., 8-bromo guanosine; deaza nucleotides, e.g., 7-deaza-adenosine; and N-alkylated nucleotides, e.g., N6-methyl adenosine. Also, sugar-modified ribonucleotides can have the 2′—OH group replaced by an H, alkoxy (or OR), R or alkyl, halogen, SH, SR, amino (such as NH₂, NHR, NR₂), or CN group, wherein R is lower alkyl, alkenyl, or alkynyl. In some embodiments, modified ribonucleotides can have the phosphodiester group connecting to adjacent ribonucleotides replaced by a modified group, such as a phosphorothioate group.

Modified sugars can include D-ribose, 2′-O-alkyl (including 2′-O-methyl and 2′-O-ethyl), i.e., 2′-alkoxy, 2′-amino, 2′-S-alkyl, 2′-halo (including 2′-fluoro), 2′-methoxyethoxy, 2′-allyloxy (—OCH₂CH═CH₂), 2′-propargyl, 2′-propyl, 2′ amine, ethynyl, ethenyl, propenyl, and cyano and the like. The sugar moiety can also be a hexose.

The term “hydrophobic modifications” refers to modification of bases such that overall hydrophobicity is increased and the base is still capable of forming close to regular Watson-Crick interactions. Non-limiting examples of base modifications include 5-position uridine and cytidine modifications like phenyl, 4-pyridyl, 2-pyridyl, indolyl, and isobutyl, phenyl (C₆H₅OH); tryptophanyl (C₈H₆N)CH₂CH(NH₂)CO), Isobutyl, butyl, aminobenzyl; phenyl; and naphthyl.

In some aspects, oligonucleotides of the invention comprise 3′ and 5′ termini. The 3′ and 5′ termini of an oligonucleotide can be substantially protected from nucleases, for example, by modifying the 3′ or 5′ linkages (e.g., U.S. Pat. No. 5,849,902 and WO 98/13526). Oligonucleotides can be made resistant by the inclusion of a “blocking group.” The term “blocking group” as used herein refers to substituents (e.g., other than OH groups) that can be attached to oligonucleotides or nucleomonomers, either as protecting groups or coupling groups for synthesis (e.g., FITC, propyl (CH₂—CH₂—CH₃), glycol (—O—CH₂—CH₂—O—) phosphate (PO₃²⁻), hydrogen phosphonate, or phosphoramidite). “Blocking groups” also include “end blocking groups” or “exonuclease blocking groups” which protect the 5′ and 3′ termini of the oligonucleotide, including modified nucleotides and non-nucleotide exonuclease resistant structures.

Exemplary end-blocking groups include cap structures (e.g., a 7-methylguanosine cap), inverted nucleomonomers, e.g., with 3′-3′ or 5′-5′ end inversions (see, e.g., Ortiagao et al. 1992. Antisense Res. Dev. 2:129), methylphosphonate, phosphoramidite, 2′-5′ internucleotide linkage non-nucleotide groups (e.g., non-nucleotide linkers, amino linkers, conjugates) and the like. The 3′ terminal nucleotide can comprise a modified sugar moiety. The 3′ terminal nucleotide comprises a 3′-O that can optionally be substituted by a blocking group that prevents 3′-exonuclease degradation of the oligonucleotide. For example, the 3′-hydroxyl can be esterified to a nucleotide through a 3′→3′ internucleotide linkage. For example, the alkyloxy radical can be methoxy, ethoxy, or isopropoxy, and preferably, ethoxy. Optionally, the 3′→3′ linked nucleotide at the 3′ terminus can be linked by a substitute linkage. To reduce nuclease degradation, the 5′ most 3′→5′ linkage can be a modified linkage, e.g., a phosphorothioate or a P-alkyloxyphosphotriester linkage. Preferably, the two 5′ most 3′→5′ linkages are modified linkages. Optionally, the 5′ terminal hydroxy moiety can be esterified with a phosphorus containing moiety, e.g., phosphate, phosphorothioate, or P-ethoxyphosphate.

In some aspects, oligonucleotides can comprise both DNA and RNA.

In some aspects, at least a portion of the oligonucleotides are linked by a substitute linkage, e.g., a phosphorothioate linkage. The presence of substitute linkages can improve pharmacokinetics due to their higher affinity for serum proteins.

The surface density of the oligonucleotides may depend on the size and type of the core and on the length, sequence and concentration of the oligonucleotides. A surface density adequate to make the nanoparticles stable and the conditions necessary to obtain it for a desired combination of nanoparticles and oligonucleotides can be determined empirically. Generally, a surface density of at least 100 oligonucleotides per particle will be adequate to provide stable core-oligonucleotide conjugates. Preferably, the surface density is at least 5, 10, 15, 20, 25, 50, 75, 100, 150, 200, 300, 400, 500, 600, 700, 800, 900, 1,000, 1,200, 1,400, 1,600, 1,800, 2,000, 5,000 or 10,000 oligonucleotides per nanoparticle.

According to another aspect of the invention a method of stimulating an immune response is provided. The method involves administering an SNA comprising a CpG immunostimulatory oligonucleotide to a subject in an amount effective to induce an immune response in the subject. Preferably the CpG immunostimulatory oligonucleotide is administered orally, locally, in a sustained release device, mucosally, systemically, parenterally, intranasally, intraocularly, or intramuscularly. When the SNA comprising CpG immunostimulatory oligonucleotide is administered to the mucosal surface it may be delivered in an amount effective for inducing a mucosal immune response or a systemic immune response.

Subject doses of the compounds described herein typically range from about 0.1 μg to 10,000 mg, more typically from about 1 μg/day to 8000 mg, and most typically from about 10 μg to 100 μg. Stated in terms of subject body weight, typical dosages range from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above. The absolute amount will depend upon a variety of factors including the concurrent treatment, the number of doses and the individual patient parameters including age, physical condition, size and weight. These are factors well known to those of ordinary skill in the art and can be addressed with no more than routine experimentation. It is preferred generally that a maximum dose be used, that is, the highest safe dose according to sound medical judgment.

The present disclosure, in other aspects, provides a method for treating cancer, including administering by intravenous, intratumoral or subcutaneous injection to a subject having cancer the invention described herein.

In some embodiments the method includes exposing the subject to an antigen wherein the immune response is an antigen-specific immune response. In some embodiments the antigen is selected from the group consisting of a tumor antigen, a viral antigen, a bacterial antigen, a parasitic antigen and a peptide antigen. In other embodiments the antigen is incorporated into the an SNA comprising the CpG oligonucleotide.

The SNA comprising the CpG immunostimulatory oligonucleotides are capable of provoking a broad spectrum of immune response. For instance these SNA comprising the CpG immunostimulatory oligonucleotides can be used to redirect a Th2 to a Th1 immune response. CpG immunostimulatory oligonucleotides may also be used to activate an immune cell, such as a lymphocyte (e.g., B and T cells), a dendritic cell, and an NK cell. The activation can be performed in vivo, in vitro, or ex vivo, i.e., by isolating an immune cell from the subject, contacting the immune cell with an effective amount to activate the immune cell of the CpG immunostimulatory oligonucleotide and re-administering the activated immune cell to the subject. In some embodiments the dendritic cell presents a cancer antigen. The dendritic cell can be exposed to the cancer antigen ex vivo.

The immune response produced by SNA comprising the CpG immunostimulatory oligonucleotides may also result in induction of cytokine production, e.g., production of IL-6, IL-8, IL-12, IL-18, TNF, IFN-α, chemokines, and IFN-γ.

In still another embodiment, the SNA comprising the CpG immunostimulatory oligonucleotides are useful for treating cancer. The CpG immunostimulatory oligonucleotides are also useful according to other aspects of the invention in preventing cancer (e.g., reducing a risk of developing cancer) in a subject at risk of developing a cancer. The cancer may be selected from the group consisting of biliary tract cancer, breast cancer, cervical cancer, choriocarcinoma, colon cancer, endometrial cancer, gastric cancer, intraepithelial neoplasms, B-cell and T-cell lymphomas, liver cancer, lung cancer (e.g. small cell and non-small cell), melanoma, neuroblastomas, oral cancer, ovarian cancer, pancreatic cancer, prostate cancer, rectal cancer, sarcomas, thyroid cancer, and renal cancer, ovarian cancer, as well as other carcinomas and sarcomas. In some important embodiments, the cancer is selected from the group consisting of bone cancer, brain and CNS cancer, connective tissue cancer, esophageal cancer, eye cancer, Hodgkin's lymphoma, Non-Hodgkin's lymphoma, larynx cancer, oral cavity cancer, melanoma and other skin cancers, and testicular cancer.

The SNA comprising the CpG immunostimulatory oligonucleotides may also be used for increasing the responsiveness of a cancer cell to a cancer therapy (e.g., an anti-cancer therapy), optionally when the CpG immunostimulatory oligonucleotide is administered in conjunction with an anti-cancer therapy. The anti-cancer therapy may be a chemotherapy, a vaccine (e.g., an in vitro primed dendritic cell vaccine or a cancer antigen vaccine) or an antibody based therapy. This latter therapy may also involve administering an antibody specific for a cell surface antigen of, for example, a cancer cell, wherein the immune response results in antibody-dependent cellular cytotoxicity (ADCC). In one embodiment, the antibody may be selected from the group consisting of Ributaxin, Herceptin, Quadramet, Keytruda, Opdivo, Bexxar, Vectibix, Arzerra, Yervoy, Zevalin, Darzalex, Erbitux, Adcetris, Avastin, Campath, Panorex, IDEC-Y2B8, BEC2, C225, Oncolym, SMART M195, ATRAGEN, Ovarex, Bexxar, LDP-03, ior t6, MDX-210, MDX-11, MDX-22, OV103, 3622W94, anti-VEGF, Zenapax, MDX-220, MDX-447, MELIMMUNE-2, MELIMMUNE-1, CEACIDE, Pretarget, NovoMAb-G2, TNT, Gliomab-H, GNI-250, EMD-72000, LymphoCide, CMA 676, Monopharm-C, 4B5, ior egf.r3, ior c5, BABS, anti-FLK-2, MDX-260, ANA Ab, SMART ID10 Ab, SMART ABL 364 Ab, ImmuRAIT-CEA, other anti-cancer antibody, including checkpoint inhibitor or antibodies that stimulate the immune system. The cancer therapy may in some embodiments be incorporated into the SNA.

Thus, according to some aspects of the invention, a subject having cancer or at risk of having a cancer is administered a CpG immunostimulatory oligonucleotide and an anti-cancer therapy. In some embodiments, the anti-cancer therapy is selected from the group consisting of a chemotherapeutic agent, an immunotherapeutic agent, a checkpoint inhibitor, immune system agonist, antibody fragment, bi-specific antibody, and a cancer vaccine.

The checkpoint inhibitor inhibits a checkpoint protein selected from the group consisting of CTLA-4, PDL1, PDL2, PD1, B7-H3, B7-H4, BTLA, HVEM, TIM3, GAL9, LAG3, VISTA, KIR, 2B4, CD160, CGEN-15049, CHK 1, CHK2, A2aR, B-7 family ligands or a combination thereof. The checkpoint inhibitor, in some embodiments, is an anti-PD-1 antibody. In some embodiments, the anti-PD-1 antibody is BMS-936558 (nivolumab). In another embodiment, the checkpoint inhibitor is an anti-CTLA-4 antibody. In other embodiments, the anti-CTLA-4 antibody is ipilimumab.

Immune system agonists may be selected from compounds that agonize CD27, CD28, B7.1, CD137, CD137L, OX40, OX40L, HVEN, and GITR.

The invention in other aspects relates to methods for preventing disease in a subject. The method involves administering to the subject a SNA comprising the CpG immunostimulatory oligonucleotide on a regular basis to promote immune system responsiveness to prevent disease in the subject. Examples of diseases or conditions sought to be prevented using the prophylactic methods of the invention include microbial infections (e.g., sexually transmitted diseases) and anaphylactic shock from food allergies.

In other aspects, the invention is a method for inducing an innate immune response by administering to the subject a SNA comprising the CpG immunostimulatory oligonucleotide in an amount effective for activating an innate immune response.

According to another aspect of the invention a method for treating or preventing a viral or retroviral infection is provided. The method involves administering to a subject having or at risk of having a viral or retroviral infection, an effective amount for treating or preventing the viral or retroviral infection of any of the compositions of the invention. In some embodiments the virus is caused by a hepatitis virus e.g., hepatitis B, hepatitis C, HIV, herpes virus, or papillomavirus.

A method for treating or preventing a bacterial infection is provided according to another aspect of the invention. The method involves administering to a subject having or at risk of having a bacterial infection, an effective amount for treating or preventing the bacterial infection of any of the compositions of the invention. In one embodiment the bacterial infection is due to an intracellular bacteria.

In another aspect the invention is a method for treating or preventing a parasite infection by administering to a subject having or at risk of having a parasite infection, an effective amount for treating or preventing the parasite infection of any of the compositions of the invention. In one embodiment the parasite infection is due to an intracellular parasite. In another embodiment the parasite infection is due to a non-helminthic parasite.

In some embodiments the subject is a human and in other embodiments the subject is a non-human vertebrate selected from the group consisting of a dog, cat, horse, cow, pig, turkey, goat, fish, monkey, chicken, rat, mouse, and sheep.

In another aspect the invention relates to a method for inducing a Th1 immune response by administering to a subject any of the compositions of the invention in an effective amount to produce a Th1 immune response.

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Those skilled in the art will recognize, or be able to ascertain using no more than routine experimentation, many equivalents to the specific embodiments of the invention described herein. Such equivalents are intended to be encompassed by the following claims.

All references, including patent documents, disclosed herein are incorporated by reference in their entirety.

### Example

### Characterization of Novel Nucleic Acid-Based TLR9 Agonists

### Background

Spherical nucleic acids (SNAs) contain densely packed oligonucleotides radially oriented on a spherical lipid bilayer. This architecture gives SNAs unique properties, including enhanced cellular uptake and endosomal receptor activation kinetics, compared with linear oligonucleotides.

The characteristics of nucleic-acid based TLR9 agonists in SNA format are described. Oligonucleotides with varying lengths and number of CpG motifs were characterized using reporter cells, human PBMCs, and in vivo in mice. Unlike linear CpG oligonucleotides, CpG oligonucleotides as short as 12-mers were tested in SNA format and found to activate TLR9 and do not require a 5′-TCG motif. TLR9 agonist SNAs retain high specificity for TLR9, increase uptake into human PBMCs, and induce increased Th1-type cytokines compared with linear CpG oligonucleotides. Potent cytokine induction was observed in mice following systemic administration. SNA architecture reduces the length and motif requirements for synthetic TLR9 agonist oligonucleotides without impairing TLR9 specificity and increases cellular uptake. These distinctive properties of SNAs underscore the utility of immunostimulatory SNAs for therapeutic applications.

### Results

### NF-κB Activation in Reporter Cells

The TLR9-stimulating activity of the nucleic acids was assessed in NF-κB reporter cell lines derived from human Ramos B-lymphocytes and mouse RAW macrophages (Table 1). SNA constructs were more potent than the same sequence in linear format. Sequences that poorly stimulated TLR9 as linear oligonucleotides due to shorter lengths (for example, see sequence 6) or the lack of 5′ TCG (for examples, see sequence 2 and sequence 4) were highly active as SNAs. Both B-class and C-class CpG were active in SNA format. To confirm that NF-κB activation is TLR9-dependent, similar experiments were performed in HEK cell lines with no exogenous TLR expression (null), or stably transformed to express human TLR9, mouse TLR9, human TLR3, human TLR7, or human TLR8. Only TLR9-expressing cell lines responded to treatment with CpG oligonucleotides.

TABLE 1
TLR9-dependent NF-κB activation

| | fold reference 8-class CpG SNA at 0.3-0.5 μM oligo | | | |
|---|---|---|---|---|
| B-class CpG | hTLR9 | mTLR9 | RAMOS | RAW |
| Reference B-class CpG SNA | 1 | 0.08 | 1 | 0.34 |
| Reference B-class CpG linear | 0.95 | n.d. | 1.52 | n.d. |
| Reference B-class CpG SNA | 0.08 | 1 | 0.17 | 1 |
| (mouse) | | | | |
| Reference B-class CpG linear | 0.17 | 3.38 | 0.86 | n.d. |
| (mouse) | | | | |
| Sequence 1 SNA | n.d. | n.d. | n.d. | n.d. |
| Sequence 2 SNA | 0.80 | n.d. | n.d. | n.d. |
| Sequence 2 linear | 0.36 | n.d. | n.d. | n.d. |
| Sequence 3 SNA | 0.08 | n.d. | n.d. | n.d. |
| Sequence 3 linear | 0.17 | n.d. | n.d. | n.d. |
| Sequence 4 SNA | 0.71 | 0.08 | 2.77 | 0.13 |
| Sequence 4 linear | 0.19 | n.d. | n.d. | n.d. |
| Sequence 5 SNA | 1.05 | 0.08 | 1.29 | 0.31 |
| Sequence 6 SNA | 0.84 | 0.08 | 1.6 | n.d. |
| Sequence 6 linear | 0.11 | n.d. | n.d. | n.d. |
| Sequence 7 SNA | 0.13 | n.d. | n.d. | n.d. |
| Sequence 7 linear | 0.11 | n.d. | n.d. | n.d. |
| Sequence 8 SNA | 0.15 | n.d. | n.d. | n.d. |
| Sequence 8 linear | 0.05 | n.d. | n.d. | n.d. |
| Sequence 9 SNA | 0.08 | 0.18 | 0.08 | 0.12 |
| Sequence 10 SNA (mouse) | 0.08 | 0.30 | 0.75 | 1.00 |
| Sequence 10 linear (mouse) | 0.08 | 0.08 | n.d. | 0.58 |
| Sequence 11 SNA | 0.08 | n.d. | n.d. | n.d. |
| Sequence 11 linear | 0.08 | n.d. | n.d. | n.d. |
| Sequence 12 SNA | 0.13 | n.d. | n.d. | n.d. |
| Sequence 12 linear | 0.13 | n.d. | n.d. | n.d. |
| Sequence 13 SNA | 0.13 | n.d. | n.d. | n.d. |
| Sequence 13 linear | 0.13 | n.d. | n.d. | n.d. |
| Sequence 14 SNA | 0.72 | 0.20 | 2.65 | 0.44 |
| Sequence 15 SNA | n.d. | 0.19 | 0.51 | 0.41 |
| Sequence 14 duplex SNA | 0.82 | n.d. | 2.00 | n.d. |
| Sequence 15 duplex SNA | n.d. | n.d. | n.d. | n.d. |
| Reference B-class CpG | n.d. | n.d. | n.d. | n.d. |
| duplex SNA | | | | |
| Reference C-class CpG linear | 0.08 | 13.13 | n.d. | n.d. |
| | fold reference C-class CpG linear | | | |
| C-class CpG | hTLR9 | mTLR9 | RAMOS | RAW |
| Reference C-class CpG linear | 0.625 | 0.625 | 5-10 | 5 |
| μM: | | | | |
| Reference C-class CpG linear | 1 | 1 | 1 | n.d. |
| Reference C-class CpG SNA | 0.08 | n.d. | n.d. | n.d. |
| Sequence 16 SNA | 0.08 | 0.08 | 1.50 | 0.20 |
| Sequence 16 linear | 0.08 | 0.29 | 2.10 | 0.78 |
| Sequence 17 SNA | 0.09 | 0.08 | 2.80 | 0.58 |
| Sequence 17 linear | 0.08 | 1.19 | 2.90 | 1.26 |
| Sequence 18 SNA | 0.09 | n.d. | n.d. | n.d. |
| Sequence 19 SNA | 0.08 | n.d. | n.d. | n.d. |
| Sequence 20 SNA | 0.09 | n.d. | n.d. | n.d. |
| Sequence 14 duplex SNA | n.d. | n.d. | n.d. | n.d. |
| Sequence 15 duplex SNA | 1.25 | n.d. | n.d. | n.d. |
| Sequence 16 duplex SNA | 0.09 | n.d. | n.d. | n.d. |
| Sequence 17 duplex SNA | 0.08 | n.d. | n.d. | n.d. |
| Reference B-class CpG | 0.09 | n.d. | n.d. | n.d. |
| duplex SNA | | | | |

n.d. = not determined
No activation was seen in null, hTLR3, hTLR7, or hTLR8 reporter cell lines

### Cytokine Response in Human PBMCs

The human cytokine response to nucleic acids was modeled using human peripheral blood mononuclear cells (hPBMCs) in culture. Following treatment with nucleic-acid based TLR9 agonists, the cytokine levels in hPMBC culture supernatants were quantified (Table 2). SNA constructs were more potent than the same sequence in linear format. Sequences with shorter lengths (for example, see sequence 6, sequence 9, and sequence 14) or lacking a 5′ TCG (for example, see sequence 4 and sequence 5) were more active as SNAs than as linear oligonucleotides. Sequences as short as 12 nt (see sequence 9) were highly active as SNAs. A-class, B-class, and C-class CpG were active in SNA format.

TABLE 2
Cytokine response in human PBMCs

| | pg/mL from hPBMC treated with 2.5 μM oligo | | | | | |
|---|---|---|---|---|---|---|
| B-class CpG | IFNα | IL-6 | IL-12p40 | TNFα | IFNγ | RANTES |
| Reference B-class CpG SNA | n.d. | 495 | 135 | 692 | 134 | 809 |
| Reference B-class CpG linear | n.d. | 163 | 115 | 122 | 174 | 180 |
| Reference B-class CpG SNA | n.d. | 103 | 88 | 147 | 31 | 327 |
| (mouse) | | | | | | |
| Reference B-class CpG linear | n.d. | 108 | 91 | 122 | 92 | 201 |
| (mouse) | | | | | | |
| PBMC donor: 36 yo Caucasian | | | | | | |
| male | | | | | | |
| Reference B-class CpG SNA | 256 | 496 | 47 | 161 | 23 | 311 |
| Sequence 4 SNA | 60 | 345 | 59 | 60 | 23 | 311 |
| Sequence 4 linear | 88 | 161 | 56 | 63 | 23 | 311 |
| Sequence 9 SNA | 1115 | 619 | 87 | 118 | 23 | 311 |
| Sequence 9 linear | 95 | 51 | 28 | 27 | 23 | 311 |
| PBMC donor: 44 yo Hispanic | | | | | | |
| male | | | | | | |
| Reference B-class CpG SNA | 62 | 312 | 39 | 302 | 65 | 1992 |
| Sequence 5 SNA | 62 | 441 | 51 | 143 | 65 | 589 |
| Sequence 5 linear | 62 | 355 | 30 | 107 | 65 | 311 |
| Sequence 6 SNA | 146 | 650 | 59 | 155 | 65 | 649 |
| Sequence 6 linear | 62 | 202 | 25 | 83 | 65 | 307 |
| PBMC donor: 22 yo African | | | | | | |
| American male | | | | | | |
| Reference B-class CpG SNA | 39 | 335 | 47 | 256 | 40 | 2778 |
| Sequence 14 SNA | 192 | 507 | 73 | 175 | 39 | 929 |
| Sequence 14 linear | 21 | 221 | 9 | 80 | 22 | 347 |
| Sequence 15 SNA | 129 | 420 | 50 | 226 | 58 | 1218 |
| Sequence 15 linear | 21 | 242 | 26 | 124 | 22 | 465 |
| PBMC donor: 22 yo African | | | | | | |
| American male | | | | | | |
| C-class CpG | IFNα | IL-6 | IL-12p40 | TNFα | IFNγ | RANTES |
| | pg/mL from hPBMC treated with 0.37 μM oligo | | | | | |
| Reference C-class CpG SNA | 1300 | 1205 | 44 | 117 | 153 | 2636 |
| Reference C-class CpG linear | 912 | 463 | 54 | 67 | 26 | 2800 |
| PBMC Donor: 26 yo Hispanic | | | | | | |
| male | | | | | | |
| | pg/mL from hPBMC treated with 0.22 μM oligo | | | | | |
| Reference C-class CpG linear | 4987 | 1241 | 26 | 128 | 290 | 1636 |
| Reference C-class CpG linear | 5000 | 969 | 23 | 146 | 215 | 2329 |
| duplex | | | | | | |
| Reference B-class CpG duplex | 5000 | 1936 | 31 | 241 | 425 | 2746 |
| SNA | | | | | | |
| Sequence 16 SNA | 4894 | 1079 | 17 | 155 | 335 | 1723 |
| Sequence 16 linear | 5000 | 957 | 14 | 114 | 211 | 1111 |
| Sequence 17 SNA | 5000 | 1908 | 77 | 151 | 359 | 2753 |
| Sequence 17 linear | 4000 | 1700 | 77 | 129 | 189 | 2800 |
| Sequence 18 SNA | 4975 | 1319 | 25 | 131 | 316 | 1373 |
| Sequence 18 linear | 3032 | 1011 | 23 | 121 | 85 | 1048 |
| Sequence 19 SNA | 3926 | 1137 | 24 | 210 | 333 | 2179 |
| Sequence 19 linear | 2904 | 884 | 15 | 113 | 156 | 1010 |
| Sequence 20 SNA | 861 | 1521 | 77 | 174 | 187 | 2800 |
| Sequence 20 linear | 4214 | 1341 | 77 | 189 | 171 | 2553 |
| Sequence 16 duplex SNA | 1558 | 1000 | 23 | 182 | 238 | 1455 |
| Sequence 16 duplex linear | 5000 | 1455 | 23 | 178 | 286 | 2800 |
| Sequence 17 duplex SNA | 1482 | 355 | 77 | 95 | 106 | 1054 |
| Sequence 17 duplex linear | 1715 | 246 | 77 | 83 | 59 | 2800 |
| PBMC donor: 30 yo African | | | | | | |
| American male | | | | | | |
| | pg/mL from hPBMC treated with 0.125 μM oligo | | | | | |
| A-class CpG | IFNα | IL-6 | IL-12p40 | TNFα | IFNγ | RANTES |
| Reference A-class CpG SNA | 4067 | n.d. | n.d. | n.d. | n.d. | n.d. |
| Reference A-class CpG linear | 1412 | n.d. | n.d. | n.d. | n.d. | n.d. |
| PBMC donor: 25 yo African | | | | | | |
| American male | | | | | | |

n.d. = not determined

### Oligonucleotide Uptake in Human PBMCs

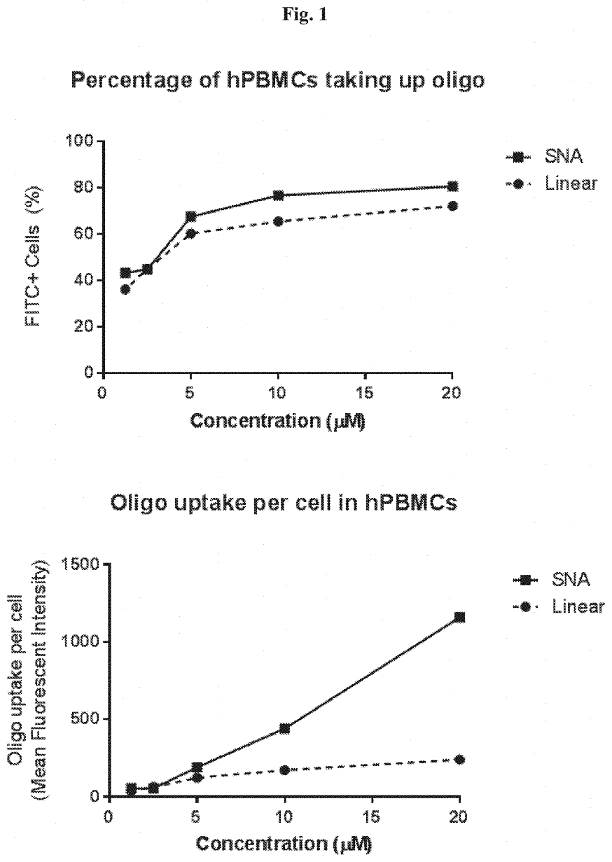

The uptake of fluorescently-labeled nucleic acids by human PBMCs was assessed by flow cytometry (FIG. 1). As compared with linear oligonucleotide, a higher percentage of cells took up SNAs and of cells that take up oligonucleotide more SNAs were taken up per cell.

### Cytokine Response In Vivo

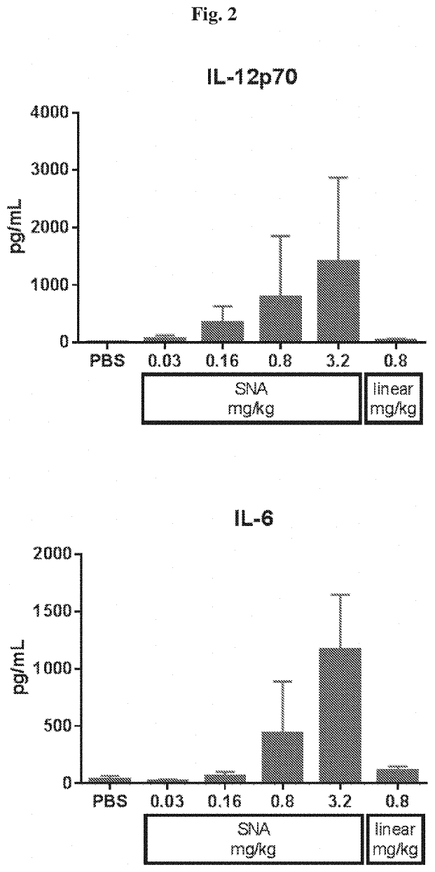

Following subcutaneous injection of CpG oligonucleotides in mice, higher serum cytokine levels were observed with SNA than linear oligonucleotide (FIG. 2).

### Materials and Methods

### SNA Preparation.

DNA oligonucleotides were synthesized with phosphorothioate (PS) internucleotide linkages. For DNA oligonucleotides in SNA format a cholesterol moiety (3′-Chol) was attached to the 3′-end via two hexaethyleneglycol (sp18) moieties. SNA compound was functionalized onto 50 nm DOPC liposomes (LSNA) at a ratio of 100 oligonucleotide molecules/liposome.

### NF-κB Activation in Reporter Cells.

NF-κB reporter cells (human Ramos B-lymphocytes, mouse RAW macrophages, human TLR9-HEK, mouse TLR9-HEK, null1-HEK, human TLR3-HEK, human TLR7-HEK, and human TLR8-HEK; Invivogen) were cultured in growth medium composed of DMEM, 4.5 g/l glucose, 10% (v/v) fetal bovine serum, 50 U/mL penicillin, 50 μg/mL streptomycin, 100 μg/mL Normocin, 100 μg/mL Zeocin, 10 μg/mL Blasticidin, 2 mM L-glutamine. Cell cultures were stored in T75 flasks (Nonpyrogenic polystyrene) from Corning at 37° C. and 5% CO2. At 24 hours following addition of nucleic acid, NF-κB activation was assessed using QuantiBlue reagent (Invivogen). 160 μL of QuantiBlue was added to each well of a sterile 96-well plate, and 40 μL of cell supernatant was added to their corresponding well to obtain a total volume of 200 μL. Once all the test compounds were plated, the plates were placed in an incubator at 37° C. and 5% CO2 for 30 minutes. Color progression was checked every 15 minutes after the 30-minute incubation period. After development of color using the standard curve as a reference, the plate was read using a fluorescence plate reader (Synergy 4) at an absorbance of 650 nm.

### Cytokine Response in Human PBMCs.

For human peripheral blood mononuclear cell (PBMC) culture, buffy coats were purchased from Zen Bio and AllCells. Buffy coats were further processed using ammonium chloride to lyse and remove red blood cells. Cells were used fresh within 24 hours from collection. PBMC were cultured in supplemented RPMI growth media (RPMI with Phenol Red (Corning), 4.5 g/l glucose, 10% (v/v) fetal bovine serum, 50 U/ml penicillin, and 50 mg/ml streptomycin) at 37° C. and 5% CO2. At 24 hours following addition of nucleic acid, the level of cytokines in the PBMC culture supernatant was assessed using multiplex cytokine ELISA kits (Quansys). A standard curve was prepared using sample diluent, which was provided in the kit. The supernatant collected from the transfected cells were diluted 1:2 using sample diluent. 50 μL of standard and samples were added to the Q-Plex 96-well plate. The plate was sealed and placed on the shaker (500 rpm and 20° C.) for 1 hour. The plate was then washed 3 times with wash buffer. 50 μL of Detection mix was added to each well. Again, the plate was sealed and placed on shaker (500 rpm and 20° C.) for 1 hour. The plate was then washed 3 more times. 50 μL of Streptavidin-HRP 1× was added to each well, and the plate was sealed and returned to the shaker (500 rpm and 20° C.) for 15 minutes. During this time mixed substrate was prepared, taking care to protect it from UV light. The plate was then washed 6 times. 50 μL of substrate mix were added to each well, and the plate was read using a Q-View LS imager within 15 minutes.

### Oligonucleotide Uptake in Human PBMCs.

Human PBMCs were cultured as described above. PBMCs were treated with FITC-labeled oligonucleotide and 24 hrs later flow cytometry was used to assess uptake of oligonucleotide.

### Cytokine Response In Vivo.

Reference B-class CpG (human) linear oligonucleotide or SNA was injected subcutaneously into 10-week-old male C57BL/6 mice. Four hours following injection, whole blood was collected and processed to serum. Cytokines were assessed using multiplex cytokine ELISA kits as described above.

### Oligonucleotide Sequences

TABLE 3

| | | | SEQ | | SEQ |
|---|---|---|---|---|---|
| | CpG | | ID | | ID |
| Oligonucleotide | class | Linear Sequence | NO: | SNA sequence | NO: |
| Reference A- | A | G*G*GGGACGATCG | 1 | G*G*GGGACGATCGTC*G*G*G* | 39 |
| class CpG | | TC*G*G*G*G*G*G | | G*G*G/isp18//isp18//3CholTEG/ | |
| Reference B- | B | T*C*G*T*C*G*T*T*T* | 2 | T*C*G*T*C*G*T*T*T*T*G*T*C* | 40 |
| class CpG | | T*G*T*c*G*T*T*T* | | G*T*T*T*T*G*T*C*G*T*T/iSp18/ | |
| | | T*G*T*C*G*T*T | | /iSp18//3CholTEG/ | |
| Reference B- | B | T*C*G*T*C*G*T*T*T | 3 | T*C*G*T*C*G*T*T*T*T*G*T*C* | 41 |
| class CpG-RTC | | *T*G*T*C*G*T*T*T* | | G*T*T*T*T*G*T*C*G*T*/FluorT// | |
| | | T*G*T*C*G*T*/Fluor | | iSp18//iSp18//3CholTEG/ | |
| | | T/ | | | |
| Reference B- | B | T*C*C*A*T*G*A*C* | 4 | T*C*C*A*T*G*A*C*G*T*T*C*C | 42 |
| class CpG | | G*T*T*C*C*T*G*A* | | *T*G*A*C*G*T*T/iSp18//iSp18// | |
| (mouse) | | C*G*T*T | | CholTEG/ | |
| Reference C- | C | T*C*G*T*C*G*T*T*T* | 5 | T*C*G*T*C*G*T*T*T*T*C*G*G* | 43 |
| class CpG | | T*C*G*G*C*G*C*G* | | C*G*C*G*C*G*C*C*G/isp18//isp18// | |
| | | C*G*C*C*G | | 3CholTEG/ | |
| Sequence 1 | B | C*G*T*C*G*T*T*T*T* | 6 | C*G*T*C*G*T*T*T*T*G*T*C*G* | 44 |
| | | G*T*C*G*T*T*T*T* | | T*T*T*T*G*T*C*G*T*T/iSp18// | |
| | | G*T*C*G*T*T | | iSp18//3CholTEG/ | |
| Sequence 2 | B | G*T*C*G*T*T*T*T* | 7 | G*T*C*G*T*T*T*T*G*T*C*G*T* | 45 |
| | | G*T*C*G*T*T*T*T* | | T*T*T*G*T*C*G*T*T/iSp18//iSp18// | |
| | | G*T*c*G*T*T | | 3CholTEG/ | |
| Sequence 3 | B | C*G*T*T*T*T*G*T*C* | 8 | C*G*T*T*T*T*G*T*C*G*T*T*T* | 46 |
| | | G*T*T*T*T*G*T*C* | | T*G*T*C*G*T*T/iSp18//iSp18// | |
| | | G*T*T | | 3CholTEG/ | |
| Sequence 4 | B | G*T*T*T*T*G*T*C* | 9 | G*T*T*T*T*G*T*C*G*T*T*T*T* | 47 |
| | | G*T*T*T*T*G*T*C* | | G*T*C*G*T*T/iSp18//iSp18//3Chol | |
| | | G*T*T | | TEG/ | |
| Sequence 5 | B | G*T*C*G*T*T*T*T* | 10 | G*T*C*G*T*T*T*T*G*T*C*G*T* | 48 |
| | | G*T*C*G*T*T | | T/iSp18//iSp18//3CholTEG/ | |
| Sequence 6 | B | T*C*G*T*T*T*T*G*T* | 11 | T*C*G*T*T*T*T*G*T*C*G*T*T/ | 49 |
| | | C*G*T*T | | iSp18//iSp18//3CholTEG/ | |
| Sequence 7 | B | C*G*T*T*T*T*G*T*C* | 12 | C*G*T*T*T*T*G*T*C*G*T*T/ | 50 |
| | | G*T*T | | iSp18//iSp18//3CholTEG/ | |
| Sequence 8 | B | G*T*T*T*T*G*T*C* | 13 | G*T*T*T*T*G*T*C*G*T*T/iSp18// | 51 |
| | | G*T*T | | iSp18//3CholTEG/ | |
| Sequence 9 | B | T*C*G*T*C*G*T*T*T* | 14 | T*C*G*T*C*G*T*T*T*T*T*T/isp18// | 52 |
| | | T*T*T | | isp18//3CholTEG/ | |
| Sequence 10 | B | T*C*G*A*C*G*T*T* | 15 | T*C*G*A*C*G*T*T*T*T*T*T/isp18// | 53 |
| (mouse) | | T*T*T*T | | isp18//3CholTEG/ | |
| Sequence 11 | B | T*c*G*T*T*T*T*T*T* | 16 | T*C*G*T*T*T*T*T*T*T*C*G*T*T/ | 54 |
| | | T*C*G*T*T | | iSp18//iSp18//3CholTEG/ | |
| Sequence 12 | B | T*C*G*T*T*T*T*T*T* | 17 | T*C*G*T*T*T*T*T*T*T*T*T*T*T/ | 55 |
| | | T*T*T*T*T | | iSp18//iSp18//3CholTEG/ | |
| Sequence 13 | B | T*T*T*T*T*T*C*G*T* | 18 | T*T*T*T*T*T*C*G*T*T*T*T*T*T/ | 56 |
| | | T*T*T*T*T | | iSp18//iSp18//3CholTEG/ | |
| Sequence 14 | B | T*C*G*T*C*G*T*T* | 19 | T*C*G*T*C*G*T*T*C*G*T*C*G* | 57 |
| | | C*G*T*C*G*T*T*A | | T*T*A/iSp18//iSp18//3CholTEG/ | |
| Sequence 15 | B | T*C*G*T*C*G*T*T* | 20 | T*C*G*T*C*G*T*T*C*G*T*T*C* | 58 |
| | | C*G*T*T*C*G*T*T* | | G*T*T*C*G*T*T*A/iSp18//iSp18// | |
| | | C*G*T*T*A | | 3CholTEG/ | |
| Sequence 16 | C | T*C*G*T*C*G*T*C* | 21 | T*C*G*T*C*G*T*C*G*T*T*C*G* | 59 |
| | | G*T*T*C*G*T*C*G* | | T*C*G*A*C*G*A*A*C*G*A/isp18// | |
| | | A*C*G*A*A*C*G*A | | isp18//3CholTEG/ | |
| Sequence 17 | C | T*C*G*T*C*G*T*T*T* | 22 | T*C*G*T*C*G*T*T*T*T*C*G*C* | 60 |
| | | T*C*G*C*G*G*C*G* | | G*G*C*G*C*C*G*C*G/isp18// | |
| | | C*C*G*C*G | | isp18//3CholTEG/ | |
| Sequence 18 | C | T*C*G*T*C*G*T*C* | 23 | T*C*G*T*C*G*T*C*G*T*T*C*G* | 61 |
| | | G*T*T*C*G*A*A*C* | | A*A*C*G*A*C*G*A*C*G/isp18// | |
| | | G*A*C*G*A*C*G | | isp18//3CholTEG/ | |
| Sequence 19 | C | T*C*G*T*C*G*T*C* | 24 | T*C*G*T*C*G*T*C*G*T*C*G*C* | 62 |
| | | G*T*C*G*C*G*T*T* | | G*T*T*T*T*C*G*C*G*A*C*G* | |
| | | T*T*C*G*C*G*A*C* | | A*C*G*T/isp18//isp18//3CholTEG/ | |
| | | G*A*C*G*T | | | |
| Sequence 20 | C | T*C*G*T*C*G*T*C* | 25 | T*C*G*T*C*G*T*C*G*T*C*G*C* | 63 |
| | | G*T*C*G*C*G*G*A* | | G*G*A*A*C*G*C*G*A*C*G*A* | |
| | | A*C*G*C*G*A*C*G* | | C*G*T/isp18//isp18//3CholTEG/ | |
| | | A*C*G*T | | | |
| Sequence 21 | B | T*C*G*T*T*T*T*G*T* | 26 | T*C*G*T*T*T*T*G*T*C*G*T*T* | 64 |
| | | C*G*T*T*T*T*G*T* | | T*T*G*T*c*G*T*T/sp18//isp18// | |
| | | C*G*T*T | | 3CholTEG/ | |
| Sequence 22 | B | T*T*T*T*G*T*C*G*T* | 27 | T*T*T*T*G*T*C*G*T*T*T*T*G* | 65 |
| | | T*T*T*G*T*C*G*T* | | T*C*G*T*T/isp18//isp18// | |
| | | T | | 3CholTEG/ | |
| Sequence 23 | B | T*T*T*G*T*C*G*T*T* | 28 | T*T*T*G*T*C*G*T*T*T*T*G*T* | 66 |
| | | T*T*G*T*C*G*T*T | | C*G*T*T/isp18//isp18// | |
| | | | | 3CholTEG/ | |
| Sequence 24 | B | T*T*G*T*C*G*T*T*T* | 29 | T*T*G*T*C*G*T*T*T*T*G*T*C* | 67 |
| | | T*G*T*C*G*T*T | | G*T*T/isp18//isp18//3CholTEG/ | |
| Sequence 25 | B | T*G*T*C*G*T*T*T*T* | 30 | T*G*T*C*G*T*T*T*T*G*T*C*G* | 68 |
| | | G*T*C*G*T*T | | T*T/isp18//isp18//3CholTEG/ | |
| Sequence 26 | B | T*T*T*T*G*T*C*G*T* | 31 | T*T*T*T*G*T*C*G*T*T/isp18// | 69 |
| | | T | | isp18//3CholTEG/ | |
| Sequence 27 | B | T*G*C*T*G*C*T*T*T* | 32 | T*G*C*T*G*C*T*T*T*T*T*T/ | 70 |
| | | T*T*T | | isp18//isp18//3CholTEG/ | |
| Sequence 28 | B | T*C*G*T*C*G*T*T*T* | 33 | T*C*G*T*C*G*T*T*T*T*TT/ | 71 |
| | | T*TT | | isp18//isp18//3CholTEG/ | |
| Sequence 29 | B | T*C*G*T*C*G*T*TT | 34 | T*C*G*T*C*G*T*TTTTT/isp18// | 72 |
| | | TTT | | isp18//3CholTEG/ | |
| Sequence 30 | B | T*G*C*T*G*C*T*T*T* | 35 | T*G*C*T*G*C*T*T*T*T*TT/isp18// | 73 |
| | | T*TT | | isp18//3CholTEG/ | |
| Sequence 31 | B | T*G*C*T*G*C*T*TT | 36 | T*G*C*T*G*C*T*TTTTT/isp18// | 74 |
| | | TTT | | isp18//3CholTEG/ | |
| Sequence 32 | B | T*G*C*T*G*C*T*T*T* | 37 | T*G*C*T*G*C*T*T*T*T*T/isp18// | 75 |
| | | T*T | | isp18//3CholTEG/ | |
| Sequence 33 | B | T*C*G*T*C*G*T*T*T* | 38 | T*C*G*T*C*G*T*T*T*T*T/isp18// | 76 |
| | | T*T | | isp18//3CholTEG/ | |

“*” denotes a phosphorothioate bond, “/iSp18/” denotes a hexa(ethylene glycol) spacer, “/3CholTEGP” denotes a cholesterol phosphoroamidite, “/FluorT/” denotes FITC-conjugated T

### EQUIVALENTS

Those skilled in the art will recognize, or be able to ascertain using no more than routine experimentation, many equivalents to the specific embodiments of the invention described herein. Such equivalents are intended to be encompassed by the following claims.

All references, including patent documents, disclosed herein are incorporated by reference in their entirety.

1.-64. (canceled)

65. A method of treating a subject having cancer or at risk of having cancer, comprising:
administering to a subject an immune system agonist of OX40 or OX40L, and a spherical nucleic acid (SNA) to treat the subject having cancer or at risk of having cancer, wherein the SNA comprises a lipid nanoparticle having an oligonucleotide shell comprised of CpG oligonucleotides positioned on the exterior of the lipid nanoparticle.

66. The method of claim 65, further comprising administering to the subject a checkpoint inhibitor.

67. The method of claim 66, wherein the checkpoint inhibitor is selected from the group consisting of a monoclonal antibody, a humanized antibody, a fully human antibody, antibody fragment, bi-specific antibody, antibody drug conjugate, a fusion protein, or a combination thereof, and a small molecule.

68. The method of claim 66, wherein the checkpoint inhibitor inhibits a checkpoint protein selected from the group consisting of CTLA-4, PDL1, PDL2, PD1, B7-H3, B7-H4, BTLA, HVEM, TIM3, GAL9, LAG3, VISTA, KIR, 2B4, CD160, CGEN-15049, CHK 1, CHK2, A2aR, B-7 family ligands or a combination thereof.

69. The method of claim 66, wherein the checkpoint inhibitor is an anti-PD-1 antibody.

70. The method of claim 69, wherein the anti-PD-1 antibody is BMS-936558 (nivolumab) or pembrolizumab.

71. The method of claim 65, wherein each CpG oligonucleotide comprises at least one phosphorothioate internucleotide linkage.

72. The method of claim 71, wherein each internucleotide linkage of each CpG oligonucleotide is a phosphorothioate internucleotide linkage.

73. The method of claim 65, wherein the lipid nanoparticle has mean diameter of 1-150 nm.

74. The method of claim 65, wherein the CpG oligonucleotides of the oligonucleotide shell are oriented radially outwards.

75. The method of claim 65, wherein the oligonucleotide shell has a density of 5-1,000 oligonucleotides per SNA.

76. The method of claim 65, wherein each CpG oligonucleotide comprises at least one phosphorothioate internucleotide linkage.

77. The method of claim 76, wherein each internucleotide linkage of each CpG oligonucleotide is a phosphorothioate internucleotide linkage.

78. The method of claim 65, wherein at least one CpG oligonucleotide comprises or consists of the nucleic acid sequence 5′-T*C*G*T*C*G*T*T*T*T*G*T*C*G*T*T*T*T*G*T*C*G*T*T-3′ (SEQ ID NO: 2), optionally wherein each CpG oligonucleotide comprises or consists of the nucleic acid sequence 5′-T*C*G*T*C*G*T*T*T*T*G*T*C*G*T*T*T*T*G*T*C*G*T*T-3′ (SEQ ID NO: 2).

79. The method of claim 65, wherein at least one CpG oligonucleotide comprises or consists of the sequence 5′-T*C*G*T*C*G*T*T*T*T*G*T*C*G*T*T*T*T*G*T*C*G*T*T-3′/HEG/HEG/TEG Cholesteryl Ester/ (SEQ ID NO: 40), optionally wherein each CpG oligonucleotide comprises or consists of the sequence 5′-T*C*G*T*C*G*T*T*T*T*G*T*C*G*T*T*T*T*G*T*C*G*T*T-3′/HEG/HEG/TEG Cholesteryl Ester/ (SEQ ID NO: 40).

80. The method of claim 65, wherein the cancer is selected from the group consisting of biliary tract cancer; brain cancer; breast cancer; cervical cancer; choriocarcinoma; colon cancer; endometrial cancer; esophageal cancer; gastric cancer; intraepithelial neoplasms; B-cell lymphomas; T-cell lymphomas; liver cancer; lung cancer (e.g. small cell and non-small cell); melanoma; neuroblastomas; oral cancer; ovarian cancer; pancreas cancer; prostate cancer; rectal cancer; sarcomas; skin cancer; testicular cancer; thyroid cancer; and renal cancer.

81. The method of claim 80, wherein the cancer is a sarcoma.

82. The method of claim 65, wherein administration is by intravenous, intratumoral or subcutaneous injection.

83. The method of claim 65, wherein the subject is human.

84. A method of treating a subject having cancer or at risk of having cancer, comprising:
administering to a subject an immune system agonist of OX40 or OX40L, an anti-PD-1 antibody, and a spherical nucleic acid (SNA) to treat the subject having cancer or at risk of having cancer, wherein the SNA comprises a lipid nanoparticle having an oligonucleotide shell comprised of CpG oligonucleotides positioned on the exterior of the lipid nanoparticle, such that the CpG oligonucleotides are oriented radially outwards, wherein the CpG oligonucleotides comprise the sequence 5′-T*C*G*T*C*G*T*T*T*T*G*T*C*G*T*T*T*T*G*T*C*G*T*T-3′ (SEQ ID NO: 2), wherein the CpG oligonucleotides are each conjugated to a cholesterol via a linker comprising two hexaethylene glycols, wherein the cholesterol anchors the CpG oligonucleotides to the lipids of the lipid nanoparticle, and wherein each internucleoside linkage of the CpG oligonucleotides is an internucleoside phosphorothioate linkage.
